(19) United States
(12) Patent Application Publication
(10) Pub. No.: US 2003/0186345 A1
(43) Pub. Date: Oct. 2, 2003

(54) Macromolecular enzyme substrates

(76) Inventor: Glen L. Hortin, Gaithersburg, MD (US)

Correspondence Address: NEEDLE & ROSENBERG, P.C., SUITE 1000, 999 PEACHTREE STREET, ATLANTA, GA, 30309-3915, US

(21) Appl. No.: 10/343,387

(22) PCT Filed: Jul. 31, 2001
(86) PCT No.: PCT/US01/41496

(51) Int. Cl. C12Q 1/37; C12Q 1/40

(52) U.S. Cl. 435/23; 435/22

(57) ABSTRACT

The present invention features methods for measuring the activity of an enzyme (such as a proteinase or an endosaccharidase) in a sample, using a macromolecular substrate of the enzyme. Also featured are methods for: detecting the level of peptidase activity of a proteinase; measuring amylase activity in a sample; diagnosing pancreatitis in a subject; measuring the activity of a target isoenzyme in a sample; identifying a compound that modulates the activity of a proteinase or an endosaccharidase; and identifying an antibody that modulates the activity of a proteinase or an endosaccharidase, using the macromolecular substrates provided by the present invention.

### CROSS-REFERENCE TO RELATED APPLICATIONS

This application claims benefit of priority to U.S. Ser. No. 60/221,790, filed Jul. 31, 2001.

### STATEMENT OF FEDERALLY SPONSORED RESEARCH

This invention was made with intramural support from the National Institutes of Health. The government has certain rights in the invention.

### FIELD OF THE INVENTION

This invention relates generally to measurement of enzyme activity using synthetic macromolecular enzyme substrates.

### BACKGROUND OF THE INVENTION

Artificial substrates are commonly used in a broad variety of clinical, industrial, and research assays to measure the activities of various enzymes. One significant drawback of artificial substrates, however, is that they usually are substantially smaller than the natural substrate of the enzyme for which activity is being measured. The small size of these artificial substrates often limits the accuracy and/or sensitivity of an assay employing such a substrate, thereby leading to inaccurate estimates of enzyme activity in a sample.

For example, assays to measure the activities of blood proteinases (e.g., those involved in the coagulation, fibrinolytic, kinin, and complement pathways), are commonly used to diagnose and monitor various diseases. However, proteinases often form complexes with other molecules that alter their enzymatic activity, e.g., co-factors, inhibitors, binding proteins, antibodies, or biological membranes. This phenomenon skews measurements of proteinase activities in serum or plasma by techniques that employ small artificial substrates (Mackie et al., Blood Coag. Fibrinolysis 3:589-595, 1992; Hemker et al., Thromb. Haemost. 74:134-138, 1995). Such inaccurate test results may decrease the chance that a patient receives appropriate medical treatment.

The present invention overcomes this deficit in the art by providing macromolecular substrates (macrosubstrates) for enzymes such as proteinases and endosaccharidases. The macrosubstrates contain small chromogenically- or fluorogenically-labeled enzyme substrates linked to a carrier polymer such as polyethylene glycol (PEG). Use of the macrosubstrates in the methods of the invention increases the accuracy and/or sensitivity of a broad variety of enzymatic assays.

### SUMMARY OF THE INVENTION

In a first aspect, the invention features a method of detecting a proteinase in a sample, including: (a) contacting the sample with a macrosubstrate for the proteinase, and (b) detecting the amount of macrosubstrate cleavage in the sample, whereby an increase in the amount of macrosubstrate cleavage detected in the sample, compared to the amount of macrosubstrate cleavage in a control sample lacking the proteinase, detects the proteinase in the sample.

In a second aspect, the invention features a method of measuring the activity of a proteinase in a sample, including: (a) contacting the sample with a macrosubstrate for the proteinase, and (b) measuring the amount of macrosubstrate cleavage in the sample, whereby the amount of macrosubstrate cleavage measured in the sample, compared to the amount of macrosubstrate cleavage in a control sample, measures the activity of the proteinase in the sample.

In one embodiment of the first two aspects of the invention, the proteinase is a selected from a proteinase of the coagulation pathway, a proteinase of the fibrinolytic pathway, a proteinase of the complement pathway, a proteinase of an inflammatory pathway, or a proteinase of the digestive system. For example, the proteinase may be elastase.

In another embodiment of the first two aspects of the invention, the proteinase is produced by a pathogen, such as a bacterium, a virus, or a fungus. In yet other embodiments of the first two aspects of the invention, the proteinase is activated by endotoxin, or is Human Immunodeficiency Virus (HIV) protease. In still other embodiments of the first two aspects of the invention, the control sample is negative for activity of the proteinase, or the control sample is positive for activity of the proteinase.

In a third aspect, the invention features a method of detecting the amount of peptidase activity of a proteinase in a sample, including: (a) contacting a first aliquot of the sample with a small substrate for the proteinase; (b) detecting the amount of small substrate cleavage in the first aliquot of the sample, wherein the amount of small substrate cleavage measures the amount of combined proteinase and peptidase activity of the proteinase in the first aliquot of the sample; (c) contacting a second aliquot of the sample with a macrosubstrate for the proteinase; (d) detecting the amount of macrosubstrate cleavage in the second aliquot of the sample, wherein the amount of macrosubstrate cleavage measures the amount of proteinase activity of the proteinase in the second aliquot of the sample; and (e) comparing the amount of combined proteinase and peptidase activity in the first aliquot of the sample with the amount of proteinase activity in the second aliquot of the sample, wherein the difference between the amount of combined proteinase and peptidase activity in the first aliquot of the sample and the amount of proteinase activity in the second aliquot of the sample detects the amount of peptidase activity of the proteinase in the sample.

In one embodiment of the third aspect of the invention, the proteinase in the sample is bound by a proteinase inhibitor, for example, α₂-macroglobulin.

In a fourth aspect, the invention features a method of detecting an endosaccharidase in a sample, including: (a) contacting the sample with a macrosubstrate for the endosaccharidase, and (b) detecting the amount of macrosubstrate cleavage in the sample, whereby an increase in the amount of macrosubstrate cleavage detected, in the sample, compared to the amount of macrosubstrate cleavage in a control sample lacking the endosaccharidase, detects the endosaccharidase in the sample.

In a fifth aspect, the invention features a method of measuring the activity of an endosaccharidase in a sample, including: (a) contacting the sample with a macrosubstrate for the endosaccharidase, and (b) measuring the amount of macrosubstrate cleavage in the sample, whereby the amount of macrosubstrate cleavage detected in the sample, compared to the amount of macrosubstrate cleavage in a control sample, measures the activity of the endosaccharidase in the sample.

In one embodiment of the fifth aspect of the invention, the endosaccharidase is amylase. In other embodiments of the fifth aspect of the invention, the control sample is negative for activity of the endosaccharidase or the control sample is positive for activity of the endosaccharidase.

In a sixth aspect, the invention features a method of detecting amylase in a sample, including: (a) contacting the sample with an amylase macrosubstrate, and (b) detecting the amount of amylase macrosubstrate cleavage in the sample, whereby an increase in the amount of amylase macrosubstrate cleavage detected in the sample, compared to the amount of amylase macrosubstrate cleavage in a control sample lacking amylase, detects amylase in the sample.

In a seventh aspect, the invention features a method of measuring amylase activity in a sample, including: (a) contacting the sample with an amylase macrosubstrate, and (b) measuring the amount of amylase macrosubstrate cleavage in the sample, whereby the amount of amylase macrosubstrate cleavage measured in the sample, compared to the amount of amylase macrosubstrate cleavage in a control sample, measures the amylase activity in the sample.

In various embodiments of the seventh aspect of the invention, the control sample is negative for amylase activity or the control sample is positive for amylase activity.

In an eighth aspect, the invention features a method of diagnosing pancreatitis in a subject, including: (a) contacting a sample from the subject with an amylase macrosubstrate, and (b) measuring the amount of amylase macrosubstrate cleavage in the sample, whereby an increase in amylase macrosubstrate cleavage, relative to the amount of amylase macrosubstrate cleavage in a sample from a normal subject, diagnoses pancreatitis in the subject.

In a ninth aspect, the invention features a method of detecting a target isoenzyme in a sample, including: (a) contacting the sample with an antibody that specifically binds to and inhibits the activity of a background isoenzyme; (b) contacting the sample with a macrosubstrate for the target isoenzyme; and (c) detecting the amount of macrosubstrate cleavage in the sample, whereby an increase in the amount of macrosubstrate cleavage detected in the sample, compared to the amount of macrosubstrate cleavage in a control sample lacking the target isoenzyme, detects the target isoenzyme in the sample.

In a tenth aspect, the invention features a method of measuring the activity of a target isoenzyme in a sample, including: (a) contacting the sample with an antibody that specifically binds to and inhibits the activity of a background isoenzyme; (b) contacting the sample with a macrosubstrate for the target isoenzyme; and (c) measuring the amount of macrosubstrate cleavage in the sample, whereby the amount of macrosubstrate cleavage detected in the sample, compared to the amount of macrosubstrate cleavage in a control sample, measures the activity of the target isoenzyme in the sample.

In various embodiments of the tenth aspect of the invention, the control sample is negative for activity of the target isoenzyme or the control sample is positive for activity of the target isozyme. In other embodiments of the tenth aspect of the invention, the isoenzyme may be endosaccharidase, for example, pancreatic amylase, or a proteinase.

In an eleventh aspect, the invention features a method of identifying a compound that modulates the activity of a proteinase, including: (a) exposing the proteinase to a macrosubstrate and to the compound, wherein the compound does not significantly bind the macrosubstrate; and (b) measuring the activity of the proteinase, whereby an increase or a decrease in the amount of macrosubstrate cleaved by the proteinase, relative to the amount of macrosubstrate cleaved by the proteinase not exposed to the test compound, identifies a compound that modulates the activity of the proteinase.

In various embodiments of the eleventh aspect of the invention, the activity of the proteinase is increased or decreased by the compound, and/or the compound may be an antibody or an aptamer.

In a twelfth aspect, the invention features a method of identifying a compound that modulates the activity of an endosaccharidase, including: (a) exposing the endosaccharidase to a macrosubstrate and to the compound, wherein the compound does not specifically bind the macrosubstrate; and (b) measuring the activity of the endosaccharidase, whereby an increase or a decrease in the amount of macrosubstrate cleaved by the endosaccharidase, relative to the amount of macrosubstrate cleaved by the endosaccharidase not exposed to the compound, identifies a compound that modulates the activity of the endosaccharidase.

In various embodiments of the twelfth aspect of the invention, the activity of the endosaccharidase is increased or decreased by the compound, and/or the compound may be an antibody or an aptamer.

In a thirteenth aspect, the invention features a method of identifying an antibody that inhibits the activity of a proteinase, including: (a) exposing the proteinase to a macrosubstrate and to the antibody, wherein the antibody does not specifically bind the macrosubstrate; and (b) measuring the amount of macrosubstrate cleavage by the proteinase, whereby a decrease in the amount of macrosubstrate cleavage, compared to the amount of macrosubstrate cleavage by the proteinase not exposed to the antibody, identifies an antibody that inhibits the activity of the proteinase.

In a fourteenth aspect, the invention features a method of identifying an antibody that inhibits the activity of an endosaccharidase, including: (a) exposing the endosaccharidase to a macromolecular substrate and to the antibody, wherein the antibody does not specifically bind the macrosubstrate; (b) and measuring the amount of macrosubstrate cleavage by the endosaccharidase, wherein a decrease in the amount of macrosubstrate cleavage, compared to the amount of macrosubstrate cleavage by the endosaccharidase not exposed to the antibody, identifies an antibody that inhibits the activity of the endosaccharidase.

In various embodiments of the first through fifth, and ninth and tenth aspects of the invention, the sample may be a pharmaceutical preparation, a research reagent, or a foodstuff.

In a fifteenth aspect, the invention features a method of measuring the amount of heparin activity in a sample, including: (a) contacting the sample with a macrosubstrate for thrombin or factor Xa; and (b) detecting the amount of macrosubstrate cleavage in the sample, whereby the amount of macrosubstrate cleavage measured in the sample, compared to the amount of macrosubstrate cleavage in a control sample having a known amount of heparin activity measures the amount of heparin activity in the sample.

In a sixteenth aspect, the invention features a method of measuring the amount of antithrombin III activity in a sample, including: (a) contacting the sample with a macrosubstrate for thrombin or factor Xa; and (b) detecting the amount of macrosubstrate cleavage in the sample, whereby the amount of macrosubstrate cleavage measured in the sample, compared to the amount of macrosubstrate cleavage in a control sample having a known amount of antithrombin III activity measures the amount of antithrombin III activity in the sample.

In a seventeenth aspect, the invention features a method of measuring the amount of alpha-2-antiplasmin activity in a sample, including: (a) contacting the sample with a macrosubstrate for plasmin, and (b) detecting the amount of macrosubstrate cleavage in the sample, whereby the amount of macrosubstrate cleavage measured in the sample, compared to the amount of macrosubstrate cleavage in a control sample having a known amount of alpha-2-antiplasmin activity measures the amount of alpha-2-antiplasmin activity in the sample.

In an eighteenth aspect, the invention features a method of inhibiting the activity of a proteinase, comprising contacting the proteinase with a macroinhibitor, thereby inhibiting the activity of the proteinase.

In a preferred embodiment of the first through tenth, and fifteenth through seventeenth aspects of the invention, the sample is from a patient or subject.

In various preferred embodiments of the first, second, third, ten, eleventh, and eighteenth aspects of the invention, the proteinase can be a serine protease, an asparty1 protease, a cysteine protease, a metalloprotease, or a proteasome protease.

In this specification and in the claims that follow, reference is made to a number of terms which shall be defined to have the following meanings:

As used in the specification and the appended claims, the singular forms “a,” “an” and “the” include plural referents unless the context clearly dictates otherwise. Thus, for example, “a molecule” can mean a single molecule or more than one molecule.

By “about” is meant ±10% of a recited value.

By “macrosubstrate” or “macromolecular substrate” is meant a peptide or oligosaccharide that is covalently coupled to PEG or a derivative of PEG, such as methoxypolyethylene glycol (mPEG). Preferably, the macrosubstrate is also detectably labeled with a chromogen, a fluorogen, or other detectable label (e.g., a radionuclide such as ³H, ³²P ¹²⁵I, or ³⁵S), such that cleavage of the macrosubstrate by its target enzyme may be readily detected.

By “macroinhibitor” is meant a peptide or oligosaccharide that is covalently coupled to PEG or to a derivative of PEG, such as mPEG. Binding of the macroinhibitor to its target enzyme inhibits activity (e.g., substrate cleavage) of the enzyme.

By “sample” is meant any specimen that may be tested for proteinase or endosaccharidase activity or in which proteinase or endosaccharidase activity may be measured using a macrosubstrate of the invention. Examples of samples include, but are not limited to: a sample from a patient or subject, such as a body fluid, secretion, or excretion (e.g., blood, serum, plasma, urine, stool, cerebrospinal fluid, semen, sputum, saliva, tears, synovial fluid, and body cavity fluids, such as peritoneal, gastric, or pleural fluids or washings); a tissue obtained from a subject or a patient; a cell; a lysate (or lysate fraction) or extract derived from a cell; or a molecule derived from a cell or cellular material; a foodstuff for humans or other animals, e.g., milk or a dairy product such as cheese or yogurt, meat or a product containing meat, eggs or a product containing eggs, fish, legumes, grains, fruits, and vegetables, and products containing fish, legumes, grains, fruits or vegetables.

A sample may also be a pharmaceutical preparation, such as a medicament for oral ingestion or a medicament for parenteral injection (e.g., insulin). A sample may also be a research reagent, for example, a nutrient medium for culturing mammalian cells or an enzyme preparation for modifying a nucleic acid, in which contaminating proteinase activity would be undesirable. The macrosubstrates of the invention are useful for identifying unacceptable levels of a contaminant, such as an undesired proteinase or microorganism, in a foodstuff, pharmaceutical preparation, or research reagent. Macrosubstrates are also useful for determining the level of enzyme activity in an enzyme preparation intended for research, industrial, or clinical use.

By “control sample” is meant a specimen with a known amount of proteinase or endosaccharidase activity, which is used as a standard against which a sample having an unknown amount of proteinase or endosaccharidase activity is compared.

By “isoenzyme” or “isozyme” is meant one of a group of two or more enzymes that have the same substrate but may be differentiated by differences in amino acid sequence and tissue distribution. An example of a pair of isozymes is pancreatic amylase and salivary amylase.

By “target isoenzyme” is meant a specific isozyme for which a measurement of catalytic activity is desired. For example, pancreatic amylase is the target isozyme in amylase assays for diagnosing pancreatitis.

By “background isoenzyme” is meant an isozyme that interferes with the measurement of catalytic activity of the target isozyme, and therefore, must be inhibited (e.g., in an immunoinhibition assay) in order to measure activity of the target isozyme. For example, because salivary amylase and pancreatic amylase cleave the same substrate, salivary amylase is a background isozyme in assays for diagnosing pancreatitis.

By “modulate” is meant to alter, by increase or by decrease.

By “specifically binds,” “specifically binds to,” “specifically interacts with,” “specifically reacts with,” and similar terms is meant that a first molecule, such as an inhibitor of an enzyme (e.g., an antibody, aptamer, or macroinhibitor) or a substrate for an enzyme, preferentially associates with a second (i.e., target) molecule (e.g., an enzyme). Preferably, the first molecule does not substantially physically associate with other types of molecules similar to the target molecule.

By “expose” is meant to allow contact between an animal, cell, lysate or extract derived from a cell, or molecule derived from a cell, and a test compound.

By “test compound” is meant any molecule, be it naturally-occurring or artificially-derived, that is surveyed for its ability to modulate the activity of a proteinase or an endosaccharidase. Test compounds may include, for example, peptides, polypeptides (e.g., monoclonal or polyclonal antibodies), nucleic acids (e.g., aptamers), saccharides, synthetic organic molecules, naturally occurring organic molecules, and derivatives and components thereof.

By “endosaccharidase” is meant an enzyme that cleaves a oligosaccharide or polysaccharide chain at an internal glycosidic bond. Accordingly, the term “activity of an endosaccharidase” refers to the enzymatic activity of a endosaccharidase, i.e., its ability to cleave a substrate for the endosaccharidase.

By “proteinase” or “protease” is meant an enzyme that cleaves a protein, polypeptide, or peptide at a peptide bond. The term “proteinase” is used herein to indicate an endoproteinase, i.e., a proteinase that cleaves a protein, polypeptide or peptide at an internal peptide bond. Accordingly, the term “activity of a proteinase” refers to the enzymatic activity of a proteinase, i.e., its ability to cleave a substrate for the proteinase.

Additional advantages of the invention will be set forth in part in the description which follows, and in part will be obvious from the description, or may be leaned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The availability of small synthetic enzyme substrates has facilitated the development of enzymatic assays that are mainstays of clinical, industrial, and basic research laboratories. However, a major shortcoming of employing a small synthetic substrate in an enzymatic assay is that the use of a substrate substantially smaller than its natural counterpart often compromises the accuracy and/or sensitivity of the assay.

For example, proteinases in a complex mixture are often bound to other molecules, such as inhibitors. In such cases, assays for proteinases that employ small peptidyl substrates often result in an overestimate of proteinase activity in the sample, because steric blockage of the active site by an inhibitor, while inhibiting cleavage of a physiologically relevant protein substrate, may allow cleavage of a small peptide substrate.

A specific example of such an inhibitor is α₂-macroglobulin, a proteinase inhibitor that covalently binds to many proteinases in blood plasma. Although α₂-macroglobulin does not directly bind to the active site of proteinases, it sterically blocks the active site of proteinases to which it binds, thereby inhibiting their ability to cleave their physiological protein substrates. Because proteinases that are sterically blocked by factors such as α₂-macroglobulin often retain residual peptidolytic activity sufficient to cleave a small artificial substrate, an assay that relies on cleavage of a small artificial substrate to indicate the activity of a proteinase, a significant fraction of which is inactivated by α₂-macroglobulin, may yield a falsely high result.

Described herein are synthetic macromolecular substrates (macrosubstrates) for proteinases and endosaccharidases. These macrosubstrates display a single defined cleavage specificity and simple reaction kinetics similar to those of small synthetic substrates. In general, the macrosubstrates are prepared by coupling an enzyme substrate (e.g., a peptide or oligosaccharide) to a detectable label (e.g., a chromogenic or fluorogenic label) and a biologically inert polymer such as polyethylene glycol (PEG) or a PEG derivative, for example, but not limited to, methoxypolyethylene glycol (mPEG). PEG and its derivatives are commercially available, e.g., from Shearwater Polymers (Huntsville Ala.). However, any detectable label and biologically inert polymer may be used to prepare the macrosubstrates employed in the methods described herein. By changing the size of the polymer, the macrosubstrates can be prepared in various sizes ranging from the size of small polypeptides, such as aprotinin, to the size of small proteinases, such as chymotrypsin, up to the size of proteins larger than albumin. Thus, macrosubstrates that simulate the size of natural proteinase substrates may be generated for most endoproteinases. Moreover, the polymer (e.g., PEG) component of macrosubstrates serves as a highly effective protecting group against exopeptidase action to ensure that only endoproteinase activity is measured.

PEG and its chemical derivatives have a stable and inert polymeric component that assumes an extended random-coil structure with a high excluded volume per molecular weight; moreover, a wide range of polymer lengths and PEG derivatives are readily available. The macrosubstrates described in Example I below, which contain the chromogenic label p-nitroanilide (pNA), are freely soluble in water. By contrast, some of the analogous “small” substrates linked to pNA are not freely soluble.in water, which limits their use as enzyme substrates.

In particular, mPEG derivatives are well suited for generating macrosubstrates for use in the present invention, on the basis of a number of favorable attributes, such as lack of a charge, high excluded volume, favorable solubility in water and organic solvents, and low adsorption to proteins and surfaces, as described by Harris (in: Poly(ethylene glycol) Chemistry, Ed. J. M. Harris, Plenum Press, New York, 1992, pp. 1-14). For example, use of mPEG derivatives allows the preparation of monovalent derivatives in which the site of substrate attachment is precisely defined at the end of the polymer chain.

Results described herein indicate that macrosubstrates of different sizes are likely to be optimal for different applications. For example, macrosubstrates with relatively small polymeric components of 1,000-2,000 Da have the favorable solubility characteristics of the polymeric component and have slightly higher substrate efficiency than larger macrosubstrates. Larger macrosubstrates with a polymeric component of 5,000 Da and above provide more specific measures of proteinase relative to peptidase activity due to more effective exclusion from sterically hindered sites such as proteinases complexed with α₂-macroglobulin. There may be practical problems with larger macrosubstrates with linear polymeric components of 20,000 Da and above, as high concentrations of the large macrosubstrate are viscous and the molecules appear prone to aggregation. Therefore, preferred sizes for polymeric components of macrosubstrates will generally range from about 1,000 Da to about 10,000 Da for linear polymers, whereas the upper limit for branched polymers can be higher.

Use of macrosubstrates allows better modeling of the steric factors that influence the enzymatic activity of many physiological proteinases (for example, but not limited to, complement, fibrinolytic, and coagulation factors) for which the natural substrates are proteins of substantial size. The ability to selectively measure proteinase rather than peptidase activity provides more accurate measures of the functional activity of proteinases in serum, plasma, or other biological fluids, in which α₂-macroglobulin and/or other molecules often complex with proteinases and block their specific proteinase activity, but not their peptidase activity.

Complexation with the surface of a sample vessel (e.g., the wall of a test tube or microtiter well) may also result in a reduced proteinase/peptidase activity ratio for a given proteinase. Under such circumstances, macrosubstrates provide a better measure of functional proteinase activity in a sample than do small molecular substrates.

Because use of macrosubstrates in proteinase assays decreases non-specific peptidase activity, macrosubstrates can be used to increase the sensitivity of screening assays for identifying molecules that inhibit physiological activity of a target proteinase.

Immunoinhibition Assays

Synthetic macrosubstrates may be used not only to measure proteinase activity, but to measure the activity of any enzyme that will specifically cleave a substrate attached to a macromolecule such as PEG. Use of macromolecular substrates in selected enzyme assays can improve substrate solubility, alter substrate specificity and kinetics, simplify methods for substrate and product separation for endpoint reactions, and support new detection methods such as fluorescence polarization methods.

For example, macrosubstrates may be used to increase the specificity and sensitivity of enzyme immunoinhibition assays. Such assays are used in clinical laboratories to specifically measure the enzymatic activity of a target isozyme in specimens containing multiple isozymes. In these assays, samples containing multiple isoenzymes are pre-incubated with antibodies that specifically bind to and inhibit the activity of the non-target isoenzyme(s). A substrate is then added, and the activity of the target isoenzyme is specifically measured.

As a specific example, immunoassays for the measurement of amylase activity are routinely used in the diagnosis and treatment of pancreatitis. However, an elevation in overall amylase activity is not necessarily diagnostic for pancreatitis, because amylase is also produced by the salivary gland and other tissues. Specific measurement of the pancreatic amylase isoenzyme improves the accuracy for diagnosis of pancreatic disorders. Accordingly, immunoinhibition assays that employ antibodies that bind to and block the activity of the salivary isoamylase have become the most commonly used method for measuring pancreatic amylase activity for the diagnosis of pancreatitis.

Described herein are macrosubstrates containing oligosaccharides with p-nitrophenol (pNP) at their reducing termini, i.e., macrosubstrates that serve as substrates for measurement of amylase activity. Such macrosubstrates for amylase can be used to enhance immunoinhibition assays that measure the pancreatic amylase isoenzyme. Moreover, these macrosubstrates should be relatively refractory to cleavage by macroamylase, a complex of amylase and autoantibodies against amylase, which is present in about 1%-10% of the population. Because cleavage of small amylase substrates by macroamylase can result in a false diagnosis of pancreatitis, use of macrosubstrates in amylase assays increases their accuracy for the diagnosis of pancreatitis.

Also described herein is an analysis of the effect of substrate size on immunoinhibition of amylase activity. These observations provide a general model for the effect of substrate size on the performance of immunoinhibition assays for endosaccharidases; these principles also extend to immunoinhibition assays for proteinases. Macrosubstrates for use in immunoinhibition assays can be prepared in any size, depending on the size of the polymer linked to the substrate group. As described herein, use of mPEG of 5000 Da as the polymeric component yields macrosubstrates that have size exclusion chromatography behavior similar to globular proteins of 30,000 Da, corresponding to effective hydrodynamic radii of about 24 Å (Tarvers and Church, Int. J. Pept. Protein Res. 26:539-549, 1985). The macrosubstrates thus behave as molecules only slightly smaller than amylase, which is a protein of 55,000 to 60,000 Da (Zakowski and Bruns, Crit. Rev. Clin. Lab. Sci. 21:283-322, 1985; Zakowski et al., Clin. Chem. 30:62-68, 1984). mPEG has a large hydrodynamic radius relative to molecular weight because it has an extended random coil rather than a globular structure (Squire, Methods Enzymol. 117:142-53, 1985). The macrosubstrates are substantially larger than other synthetic oligosaccharide substrates of amylase, which are calculated to have effective radii of about 9 Å for 2-chloro-p-nitrophenol-α-D-maltotrioside (G3ClpNP), 11 Å for p-nitrophenyl-α-D-maltopentaoside (G5pNP), and 12 Å for 4,6-O-ethylidene p-nitrophenyl-α-D-maltoheptaoside (EtG7pNP), based on the formula of Squire (Methods Enzymol. 117:142-53, 1985). In addition, the amylase macrosubstrates described herein are resistant to digestion by exoglycosidases.

In assays using polyclonal antibodies against amylase, titration curves for inhibition of enzyme activity were observed to shift progressively to lower concentrations when larger substrates were used. Within a polyclonal antiserum, antienzyme antibodies fall into three classes: 1) Antibodies binding to epitopes within the active site and inhibiting cleavage of all substrates; 2) Antibodies binding to epitopes near the active site and inhibiting cleavage of large substrates but not small substrates (the size of this zone will depend on substrate size); and 3) Antibodies binding to epitopes distant from the active site and exerting little steric effect on substrate access. Experiments described herein indicate that use of a substrate with an effective radius of 24 Å rather than a substrate with a 9 Å radius leads to an approximate 10-fold increase in the number of antibodies that are inhibitory. Thus, as described in Example II below, the number of Class 2 antibodies in two different antisera appears to be about 10-fold greater than the number of Class 1 antibodies. A substantial effect of substrate size was noted even within the narrower range of oligosaccharide substrates containing between three and seven glucose units.

The effects of substrate size on the inhibitory activity of two monoclonal antibodies (MABs) specific for salivary amylase are described hereinbelow. MAB 88E8 potently inhibited salivary amylase activity measured with either small or large substrates. Variation in substrate size does not alter the inhibitory effect of MAB 88E8, indicating that it binds directly to the active site of salivary amylase.

A second salivary amylase-specific monoclonal antibody, MAB 66C7, appears to represent the second class of antibodies that bind to epitopes outside the active site of the enzyme, because MAB 66C7 did not significantly inhibit salivary (or pancreatic) amylase activity using either mPEG-coupled or uncoupled substrates. However, when the very large substrate amylopectin azure was used, MAB 66C7 specifically inhibited salivary amylase activity. Therefore, MAR 66C7 appears to bind relatively far away from the active site of salivary amylase, such that only very large substrates are sterically hindered from binding to the active site.

Use of macrosubstrates in inimmunoinhibition assays, such as EMIT^{R} (enzyme multiplied immunoassay technique) and CEDIA^{R} (cloned enzyme donor immunoassay), should improve the sensitivity and specificity of these assays, because larger substrates enhance immunoinhibition, particularly for antibodies that bind distant from the active site of a target enzyme.

Moreover, use of a macrosubstrate for immunoinhibition assays can increase the number of inhibitory antibodies in a polyclonal serum by at least ten-fold, relative to the inhibition observed using small substrates. Because macrosubstrates expand the number of target epitopes on enzymes, they are also useful in screening assays to identify antibodies, aptamers, or other molecules for use as enzyme inhibitors in diagnostic or therapeutic applications.

Proteinase and endosaccharidase macrosubstrates of the invention contain a specific peptide or oligosaccharide substrate that is detectably-labeled (e.g., with a chromophore or fluorophore) and linked to a polymer such as PEG. The PEG polymer, which may be of any size, is preferably between 1,000 Da and 10,000 Da. The resulting macrosubstrate may be monovalent, divalent, or polyvalent (i.e., the polymer component may carry one, two, or several substrate groups per molecule). Examples of the components of macrosubstrates, e.g., detectable labels, polymers, small substrate component (e.g., peptides and oligosaccharides), and linkages between the polymer and the small substrate component, are provided below.

Examples of Chromophores and Fluorophores for Macrosubstrates

1. p-nitroanilide (pNA; chromogenic; enzyme activity can be measured as described in Erlanger et al., Arch. Biochem. Biophys. 95:271-278, 1961 and Svendsen et al., Thrombosis Res. 1:267-278, 1972).

2. beta-naphthylamide (2-naphthylamide; chromogenic and fluorogenic; enzyme activity can be measured as described in Lee et al., Anal. Biochem. 41:397-401, 1071 and Wagner et al., Arch. Biochem. Biophys. 197:63-72, 1979).

3. 7-amido-4-methylcoumarin (AMC; fluorogenic; enzyme activity can be measured as described in Morita et al., J. Biochem. 82:1495-1498, 1977 and Zimmerman et al., Anal. Biochem. 70:258-262, 1976).

4. p-nitrophenylalanine derivatives (absorbance change; enzyme activity can be measured as described in Richards et al., J. Biol. Chem. 265:7733-7736, 1990 and Dunn et al., Biochim. Biophys. Acta 913:122-130, 1987).

Examples of Polymers for Generating Macrosubstrates

The following are examples of polymers that may be used for generating macrosubstrates. These and other polymers are well known in the art, and are commercially available, for example, from Shearwater Polymers, Inc. (Huntville, Ala.).

1. PEG (RO—(CH₂CH₂O)ₙCH₂CH₂—OR; 3,400 mw; bifunctional carrier; i.e., two sites for attachment of substrate molecules; R is a reactive group that can be linked to a peptide).

2. mPEG (CH₃O—(CH₂CH₂O)ₙCH₂CH₂—OR; 1,000 mw; 2,000 mw; 5,000 mw; 20,000 mw; one site for attachment of a substrate molecule; R is a reactive group that can be linked to a peptide).

3. (mPEG)₂Lys (a lysine molecule carrying two mPEG molecules; each mPEG of 5,000 mw; Monfardine et al., Bioconjugate Chem. 6:62-69, 1995).

4. Pendant PEGs with 5 attachment sites (5,000 mw; Kohn et al., Macromolecules 25:4476, 1992).

5. Polyacrylic acid 5,900 mw (many attachment sites; available. e.g., from Aldrich Chemical; —(CH₂CHCOOH)ₙ)—.

Examples of Amino Group-PEG Linkages in Peptidyl Macrosubstrates

The chemical linkages set forth below can be used to generate macrosubstrates containing monovalent (e.g., mPEG), bivalent, and multivalent PEGs (R=peptide group).

1. Propionamide linkage between PEG and a peptide, by reacting a succinimidyl derivative of PEG propionic acid (SPA-PEG) with a peptide: PEG-O—CH₂—CH₂—CO—(NH—R).

2. Carboxymethylamide linkage between PEG and a peptide, by reacting a succinimidyl ester of carboxymethylated PEG (SCM-PEG) with a peptide: PEG-O—CH₂—CO—(NH—R).

3. Isourea linkage between PEG and a peptide, by reacting a benzotriazole carbonate derivative of PEG (BTC-PEG), a PEG p-nitrophenol carbonate (NPC), or a carbonyldimidazole-activated PEG (CDI-PEG) with a peptide: PEG-O—CO—(NH—R).

4. Tresyl (trifluoroethyl sulfonyl) linkage between PEG and a peptide, by reacting PEG tresylate with a peptide: PEG-O—CH₂—CH₂—(NH—R) (Nilsson and Mosbach, Methods in Enzymol. 104:56, 1984; Yoshinga and Harris, J. Bioactive Comp. Polym. 4:17, 1989; Delgado et al., Biotech. Appl. Biochem. 4:17, 1989; Dust et al., Macromolecules 23:3742, 1990; Senior et al., Biochem. Biophys. Acta 1062:77, 1991; and Klibanov et al., Biochem. Biophys. Acta 1062:142, 1991).

5. Epoxide linkage between PEG and a peptide, by reacting an epoxide derivative of PEG (EPOX-PEG) with a peptide: PEG-O—CH₂—CH(OH)—CH₂—(NH—R).

6. Urea linkage between PEG and a peptide, by reacting mNCO-PEG (mPEG-O—CH₂CH₂—N═C═O) with a peptide: PEG-NH—CO—(NH—R).

7., Succinamide linkage between PEG and a peptide: formed either by reacting SSA-PEG with amino group of peptide or succinylated peptide coupled to amino-PEG: PEG-NH—CO—CH₂—CH₂—CO—(NH—R).

Examples of Carbohydrate-PEG Linkages in Oligosaccharidyl Macrosubstrates

The chemical linkages set forth below can be used to generate macrosubstrates containing monovalent, bivalent, and multivalent PEGs (R=oligosaccharide group).

1. Isourea linkage between PEG and an oligosaccharide, by reacting mNCO-PEG (mPEG-O—CR₂CH₂—N═C═O) with an oligosaccharide: mPEG-N—CO—(OR).

2. Tresyl linkage between PEG and an olgiosaccharide, by reacting PEG tresylate with an oligosaccharide: mPEG-O—CH₂—CH₂—(OR).

Examples of Elastase Substrates and PEG Sources for Generating Elastase Macrosubstrates.

| | |
|---|---|
| AlaAlaAlapNA | SPA mPEG 2,000 |
| AlaAlaAlapNA | BTC mPEG 5,000 |
| AlaAlaAlapNA | SCM mPEG 5,000 |
| AlaAlaAlapNA | NPC mPEG 5,000 |
| AlaAlaAlapNA | epoxide 5,000 |
| AlaA1aA1apNA | SPA mPEG 5,000 |
| AlaAlaAlapNA | tresyl mPEG 5,000 |
| AlaAlaAlapNA | CDI mPEG 5,000 |
| AlaAlaAlapNA | NCO mPEG 5,000 |
| AlaAlaAlapNA | SPA mPEG 5,000 |
| AlaAlaAlapNA | SPA mPEG 20,000 |
| AlaAlaAlapNA | SPA mPEG 2,000 |
| AlaAlaAlapNA | mPEG SSA 5,000 |
| AlaAlaAlapNA | mPEG SPA 20,000 |
| AlaAlaAlapNA | mPEG SPA 1,000 |
| SucAlaAlaProAlapNA | mPEG amine 5,000 |
| SucAlaAlaAlapNA | mPEG amine 5,000 |

Examples of Chymotrypsin Substrates and PEG Sources for Generating Chymostrypsin Macrosubstrates

| | |
|---|---|
| Phe-pNA | SPA2 PEG 3,400 |
| AlaAlaPhepNA | SPA2 PEG 3,400 |
| AlaPhepNA | SPA2 PEG 3,400 |
| AlaAlaPhe AMC | mPEG SSA 5,000 |
| PhepNA | mPEG SSA 5,000 |
| PhepNA | mPEG NPC 5,000 |
| PhepNA | mPEG SPA 2,000 |
| AlaAlaPhepNA | mPEG SPA 1,000 |
| AlaAlaPhepNA | mPEG SPA 2,000 |
| AlaAlaPhepNA | mPEG SPA 5,000 |
| AlaAlaPhepNA | mPEG SPA 20,000 |
| AlaAlaPhepNA | Tresyl mPEG 5,000 |
| AlaAlaPhepNA | SSA mPEG 5,000 |
| AlAAlaPhe naphthylarnide | SPA mPEG 2,000 |
| AlaAlaPhepNA | NCO-mPEG 5,000 |
| AlaAlaPhepNA | BTC mPEG 5,000 |
| AlaAlaPhepNA | SPA mPEG 20,000 |
| AlaAlaPhepNA | Lys(mPEG 5,000)2 |
| GlyPhepNA | BTC mPEG 2,000. |
| SucAlaAlaProPhepNA | mPEG amine 5,000 |
| SucPhepNA | mPEG amine 5,000 |
| AlaAlaPhepNA | methoxy(ethoxy)2 acetic |
| | acid |
| AlaAlaPhepNA | SPA mPEG 1,000 |
| AlaAlaPhepNA | SPA mPEG 2,000 |
| AlaAlaPhepNA | polyacrylic acid 5,000 |
| AlaAlaPhepNA | pendant SPA 5,000 |
| AlaAlaPheAMC | SPA mPEG 2,000 |
| PhepNA | SPA mPEG 2,000 |

Examples of Trypsin, Thrombin, Factor Xa, Plasmin Substrates and PEG Sources for Generating Macrosubstrates

| | |
|---|---|
| betaAlaGlyArgpNA | SPA mPEG 5,000 |
| GlyArgpNA | NCO mPEG 5,000 |
| GlyArgpNA | SPA2 PEG 3,400 |
| SarProArgpNA | SPA mPEG 2,000 (Sar = sarcosine) |
| D-Ile-PheLyspNA | SPA mPEG 2,000 |
| (S-2288) D-IlePheArgpNA | SPA 3,400 |
| SarProArgpNA | SPA 3,400 |
| ArgpNA | BTC mPEG 2,000 |
| GlyArgpNA | BTC mPEG 2,000 |
| GlyArgpNA | SPA mPEG 1,000 |
| CbzLysArgpNA | BTC mPEG 2,000 (Cbz = carbobenzoxy or |
| | benzyloxycarbonyl) |
| PheVal-ArgpNA | (2-step synthesis; PheVal was added to BTC mPEG 2000, |
| | after which ArgpNA was added) |
| AlaGly-ArgpNA 2HCl | (2-step synthesis; AlaGly was added to BTC mPEG |
| | 2,000, after which the C-terminal carboxyl was activated |
| | with carbodiimide to link to ArgpNA) |
| D-PhePipArgpNA | BTC mPEG 5,000 |
| GluGlyArgpNA | BTC mPEG 5,000 |

Example of an Inhibitor of Thrombin

| | |
|---|---|
| p-Aminobenzamidine | SPA mPEG 2,000 |

Examples of Aspartic Protease Substrates and PEG Sources for Generating aspartic Protease Macrosubstrates

| | |
|---|---|
| LeuSerNO₂PheNleAlaLeuOMe TFA | SPA mPEG 2,000 |
| PheAlaAlaNO₂PhePheValLeu4- | SPA mPEG 2,000 |
| Ohmethylpyr | |
| AsnLeuValTyrNleValThrGly | SPA mPEG 2,000 |

Examples of Amylase Substrates and PEG Sources for Generating Amylase Macrosubstrates

| | |
|---|---|
| Glc5pNP | NCO mPEG 5,000 |
| EthGlc7pNP | NCO mPEG 5,000 |
| EthGlc7pNP | tresyl mPEG 5,000 |
| Glc3CINP | NCO mPEG 5,000 |
| EthGlc7pNP | NCO mPEG 5,000 |

Examples of Uses for Macrosubstrates

1. Monitoring Heparin Activity

Heparin therapy often requires monitoring to determine whether a therapeutic level of anticoagulation has been pharmaceutically achieved or to determine that clearance or neutralization of heparin has been completed before specific surgical procedures are performed (Teien and Lie, Thromb. Res. 10:399-410, 1977; Scully et al., Thromb. Res. 46:447-455, 1987; Olson et al., Methods Enzymol. 222:525-559, 1993). Assays measure the cofactor activity of heparin in stimulating the inhibition of Factor Xa or thrombin by antithrombin III. Protease activity and its degree of inhibition can be measured in a clotting assay or with a chromogenic substrate. Inhibition of Factor Xa or thrombin by α₂-macroglobulin competes with the inhibition of Factor Xa or thrombin by antithrombin III. Therefore, inhibition of the protease activity of Factor Xa or thrombin by α₂-macroglobulin interferes with the accurate measurement of unbound, active Factor Xa or thrombin using small peptide substrates, because the α₂-macroglobulin-complexed enzymes retain peptidase activity. Use of macrosubstrates provides a more accurate measure of the inhibition of proteases by antithrombin III, because use of macrosubstrates decreases peptidase activity, and therefore, only physiologically relevant enzymatic activity is detected.

2. Measurement of the Activity of Individual Coagulation Complement, and Fibrinolytic Factors and their Inhibitors

Current approaches for assessing the relative activity of the coagulation, complement, or fibrinolytic pathways in a patient involve activating a plasma sample from the patient and measuring the resulting activity of the appropriate protease (such as Factor Xa, thrombin, or plasmin) using a chromogenic substrate (see, for example, Gallimore and Friberger, Blood Rev. 5:117-127, 1991; Witt, Eur. J. Clin. Chem. Clin. Biochem. 29:355-374, 1991; Simonsson et al., U.S. Pat. No. 4,748,116; Friberger et al., Haemostasis, 7:138-145, 1978; Ranby et al., Thromb. Res. 27:743-749, 1982; Stocker et al., Folia Haematol. 115:260-264, 1988; Prasa and Sturzbecher, Throm. Res. 92:99-102, 1998; Wiman and Nilsson, Clin. Chim. Acta 128:359-366, 1983). The relative level of protease inhibitors (e.g., of coagulation, fibrinolysis, and the complement pathway) in a patient plasma sample can be determined by adding the appropriate protease to the plasma and measuring the decrease in protease activity and by comparing the effect to standards with known amounts of inhibitor. Such assays are used to monitor therapy and to evaluate coagulation factor concentrates. Commonly used assays measure plasma components, including: protein C, antithrombin III, plasminogen, plasminogen activator, plasminogen activator inhibitor, α₂-antiplasmin, coagulation factors VIII and IX, and C1 inhibitor. The use of macrosubstrates in place of small peptide substrates for assays of plasma proteases and protease inhibitors involved in the coagulation, fibrinolytic, and complement pathways provides a more accurate assessment of the relative activity of these pathways, because, unlike small peptide substrates, macrosubstrates are cleaved only by proteases that are capable of physiologically relevant protease activity, as opposed to non-physiologically peptidase activity.

3. Measurement of Thrombin Generation (Thrombin Potential) in Plasma with Slow Chromogenic Substrates as a Test for Anticoagulant Function and Risk for Thrombosis

Experimental evidence has shown that use of slow chromogenic substrates can provide a more accurate measure of anticoagulant function than standard clotting tests. However, substrate cleavage by proteases bound to α₂-macroglobulin requires complicated mathematical correction for the estimated interference (Hemker et al., Thromb. Haemost. 83:589-591, 2000; Hemker et al., Thromb. Haemost. 70:617-624, 1993).

The thrombin generation assay is performed by mixing plasma, calcium, tissue factor, and a chromogenic or fluorogenic substrate that selectively measures thrombin activity. Absorbance or fluorescence from substrate cleavage is monitored over about 30 minutes. The rate of substrate cleavage reflects the balance between thrombin formation and inactivation by inhibitors. Use of macrosubstrates to measure thrombin generation improves the current assay, because the peptidase activity of α₂-macroglobulin/thrombin complexes is minimized, thereby yielding a more direct and accurate estimate of functional thrombin activity over time, and thus, coagulant function and relative risk of thrombosis.

4. Measurement of Elastase Activity

Elastases are digestive enzymes that are both produced by the pancreas and released by white blood cells during the course of an inflammatory response. The breakdown of the lung parenchyma by elastases is a critical factor in the development of emphysema (Brown and Donaldson, Thorax 43:132-139, 1988; Smith et al., Clin. Sci. 69:17-27, 1985). There are usually high concentrations of elastase inhibitors in blood and most other biological fluids; however, free elastase may be present at sites of severe inflammation, such as an abscess site. Linking a small elastase substate to mPEG increases the efficiency of substrate cleavage more than ten-fold, thereby allowing more sensitive detection of elastase activity.

5. Measurement of Asparty1 Protease Activity

Asparty1 proteases make up a functionally diverse group of proteases that include many bacterial and fungal proteases, pepsins (which serve as digestive enzymes), renin (which is involved in blood pressure regulation), and retroviral proteases, including the human immunodeficiency virus (HIV) protease, which is a major therapeutic target for treatment of HIV infection. Substrates for these enzymes are useful for the discovery of new therapeutic agents and for monitoring the efficacy of pharmaceutical therapy. These enzymes has been difficult targets for which to produce chromogenic or fluorogenic substrates, because asparty1 proteases cleave peptides substrates having six or more residues into two approximately equal segments (Kotler et al., Proc. Natl. Acad. Sci. USA 85:4185-4189, 1988; Wang et al., Anal. Biochem. 210:351, 1993; Toth and Marshall, Int. J. Peptide Protein Res. 36:544-550, 1990). The asymmetric nature of macrosubstrate cleavage products provides new methods for detecting substrate cleavage, such as fluorescence polarization.

6. Diagnostic Typing of Microbes

Various microbes of medical interest produce a protease that is diagnostic for that particular microbe (Manafi et al. Microbiol. Rev. 55:335-348, 1991). For example, Engels et al. (J. Clin. Microbiol. 14:496-500, 1981) describes a chromogenic substrate for staphylocoagulase that allows identification of Staphyloccus aureus. The macrosubstrates of the invention are useful for identification of microbes that cause infections and disease, and use of macrosubstrates for identification of such microbes can result in more efficacious treatment for infections.

After culturing a microbe from a sample or source suspected of containing or being contaminated with a disease-causing microbe (e.g., a throat swabbing, sputum, pleural fluid, urine, blood, a wound site, or a catheter), the microbe is tested for its ability to cleave a microbe-specific macrosubstrate, i.e., a substrate of a protease whose activity is diagnostic for a specific microbe. Cleavage of a specific substrate identifies the microbe, which increases the likelihood that a patient from whom the microbe has been isolated, or who may have had contact with the microbe, will receive the most appropriate treatment.

High-throughput Screening Assays Using Macrosubstrates

Macrosubstrates are useful in high-throughput screening assays for identifying compounds that modulate (e.g., inhibit or stimulate) the activity of any proteinase or endosaccharidase. One of ordinary skill in the art will understand how to identify a compound useful for therapeutic modulation of an enzyme involved in disease or for modulation of an enzyme used in industry (e.g., foodstuff manufacturing) and/or research (e.g., nucleic acid modification), using one or more high throughput screening assay techniques analogous to those that are well knows in the art (for example, but not limited to, those described in Abriola et al., J. Biomol. Screen 4:121-127, 1999; Blevitt et al., J. Biomol. Screen 4:87-91, 2000; Hariharan et al., J. Biomol. Screen 4:187-192, 1999; Fox et al., J. Biomol. Screen 4:183-186, 1999; Burbaum and Sigal, Curr. Opin. Chem. Biol. 1:72-78, 1997; Jayasena, Clin. Chem. 45:1628-1650, 1999; and Famulok and Mayer, Curr. Top. Microbiol. Immunol. 243:123-136, 1999).

One advantage of using macrosubstrates in high-throughput screening assays to identify enzyme inhibitors is that the increased steric hindrance of macrosubstrates, relative to small substrates, expands the number of binding sites on the target enzyme that will effect enzyme inhibition, thereby increasing the sensitivity of the screening assay. Thus, a screening assay using a macrosubstrate can identify a larger number of inhibitors than a screening assay using a small substrate. In addition, macrosubstrates allow the use of detection methods, such as fluorescence polarization, that cannot be used to detect cleavage of small substrates. Use of a detection method such as fluorescence polarization in a high-throughput screening assay can increase its efficiency and/or sensitivity.

For example, elastase is a proteinase that degrades lung tissue in patients with emphysema. One of ordinary skill in the art will understand how to identify an inhibitor of elastase activity using known high-throughput screening methods. Such an inhibitor can be used, for example, to treat emphysema. A typical sample in such a high-throughput assay contains elastase, a macrosubstrate that is cleaved by elastase, and a compound (e.g., from a combinatorial library) that is to be tested for its ability to inhibit elastase activity, as well as any necessary buffers, salts, or co-factors necessary for the enzyme reaction and detection thereof A test compound that inhibits cleavage of the elastase macrosubstrate, compared to a control reaction that lacks the test compound, is identified as an inhibitor of elastase, and is subjected to clinical testing, as is known in the art, for its safety and efficacy as an anti-elastase therapeutic compound for treating emphysema.

Moreover, combinatorial libraries may be screened for macroinhibitors having the ability to inhibit the enzymatic activity of a proteinase (e.g., elastase, angiotensin-converting enzyme, renin, or HIV protease) or an endosaccharidase of interest, using methods that are known to those of ordinary skill in the art. Useful inhibitors of a proteinase or endosaccharidase inhibit enzymatic activity of the target enzyme by at least 10%, preferably by at least 25%, more preferably, by at least 30%-50%, by at least 50%-75%, or by at least 75%-98%. Screening for macroinhibitors can expand the number of binding sites that will lead to steric inhibition of the active site of the enzyme, thereby increasing the likelihood of identifying an effective inhibitor, compared to conventional screens of small substrate inhibitors.

Test Compounds

In general, compounds that modulate the activity of proteinases and endosaccharidases may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds (e.g., but not limited to, antibodies, peptides, and aptamers). Synthetic compound libraries are commercially available, e.g., from Brandon Associates (Merrimack, N.H.) and Aldrich Chemical (Milwaukee, Wis.).

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the particular target enzyme being studied should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits a particular target proteinase or endosaccharidase. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate activity of the target enzyme.

The present invention is more particularly described in the following examples which are intended as illustrative only since numerous modifications and variations thereof will be apparent to those of ordinary skill in the art.

### EXAMPLE I

Macromolecular Chromogenic Substrates that Distinguish Proteinase from Peptidase Activity

A) Materials and Methods

Reagents

Bovine trypsin was purchased from Worthington Biochemicals Co. (Freehold N.J.). Active α₂-macroglobulin was obtained from Calbiochem (La Jolla, Calif.). The substrates Ala-Ala-Phe-p-nitroanilide (pNA) and Suc-Ala-Ala-Phe-pNA were from Bachem Bioscience, Inc. (King of Prussia, Pa.). S-2288 (D-Ile-Pro-Arg-pNA) and S-2238 (D-Phe-Pip-Arg-pNA) were purchased from DiaPharma Group, Inc. (West Chester, Ohio). Human thrombin, bovine chymotrypsin, polyethylene glycol, polyethylene glycol bis amine of 3,400 molecular weight, succinyl-Ala-Ala-Pro-Phe-pNA, p-amidinophenylsulfonyl fluoride, Sephadex G-25 coarse, and proteins for use as molecular weight standards were obtained from Sigma Chemical Co. (St. Louis, Mo.). Bio-Gel P-6 and P-60 were from Bio-Rad Corp. (Richmond, Calif.). Propionic acid (PA) derivatives of methoxypolyethylene glycol (mPEG) activated as N-hydroxysuccinimide esters were produced by Shearwater Chemical (Huntsville, Ala.). Polymer size of these derivatives were estimated by the manufacturer to have an average size of 1,000, 1,800, 5,100, and 21,000 Da by gel permeation chromatography for monofunctional succinimidyl propionate derivatives, 3,200 Da for a bifunctional succinimidyl propionate derivative, and 11,000 Da for a bis(monomethylpolyethylene glycol) lysine succinimidyl propionate ester.

Preparation of PEG-substate Conjugates

Coupling of Ala-Ala-Phe-pNA was performed by dissolving substrate to a concentration of 100-200 mM in 1.2 ml dimethylformamide with 10% N-ethylmorpholine and adding 0.5-1.0 molar equivalent of mPEG active ester. The mixture was incubated for two hours at room temperature, diluted with water, and product was isolated in the void volume during gel filtration chromatography on a column of Sephadex G-25 coarse in 0.1% acetic acid. Products were lyophilized to a dry powder.

Other products were prepared by coupling reactions with diisopropylcarbodiimide using ratios of reactants to yield predominantly monofunctional products although the PEGs used for these reactions contain two potential coupling sites. Monofunctional products were desired to avoid mixtures of mono- and bifunctional products that might not have exactly the same kinetic properties as substrates. Succinyl-Ala-Ala-Pro-Phe-pNA in dimethylformamide was activated with diisopropylcarbodiimide for 10 min at room temperature and mixed with PEG bis amine in dimethylformamide. After 1 hr, the reaction mixture was chromatographed on a column of Bio-Gel P-6 in 0.1 M ammonium acetate, pH 6.0 and product was collected in the void volume. Bis-succinyl PEG was prepared by action of succinic anhydride on PEG bis amine. The bis-succinyl derivative was activated with diisopropylcarbodiimide and coupled with substrates containing free amino groups such as S-2288 and S-2238. Products were isolated by chromatography on Bio-Gel P-6. Acetylation of S-2238 was preformed with acetic anhydride.

Molecular Size Analysis

Size exclusion chromatography was performed with a Pharmacia FPLC system using a 25 ml (1×31 cm) column of Superose 12 in 140 mM NaCl, 10 mM sodium phosphate, pH 7.4/10% acetonitrile with continuous monitoring of elution at 280 nm or with gravity elution of a 2.5×26 cm column of Bio-Gel P-60 in 0.1 M ammonium acetate, pH 6.5 monitored by spectrophotometric analysis of column fractions. Primary calibration of the FPLC analysis was performed with aprotinin, carbonic anhydrase, and albumin, because catalase and ferritin had low solubility in the solvent containing aceto-nitrile. The high molecular weight standards were run in completely aqueous solution to confirm the calibration curve.

HPLC Analysis

Analysis of peptides was performed with an Alliance 2690 system (Waters Corp., Milford, Mass.) with a 996 photodiode array detector and Millenium 32 software. Separations were performed on a 4.6×250 mm large-pore octadecylsilica column, Supelcosil LC-318 from Supelco, Inc (Bellefonte, Pa.). Elution at a flow rate of 1 ml/min was performed with 0.1% trifluoroacetic acid mixed with a linear gradient of acetonitile from 5% to 75% over 25 min.

Spectrophotometric Analysis of Products

Absorption spectra of products and concentrations of substrates in water were determined with a Perkin-Elmer Lambda 4B spectrophotometer using cuvettes with a 1 cm length.

Enzyme Assays

Measurements of protease activity at 25° C. or 37° C. were performed and analyzed as previously described (Hortin and Trimpe, J. Biol. Chem. 266:6866-6871, 1991) with a Cobas FARA analyzer (Roche Diagnostic Systems, Somerville, N.J.). Reactions had a total volume of 100 μl and were monitored at 410 nm. Kinetic parameters were calculated from initial rates of reactions using Lineweaver-Burke plots. Substrate solutions were prepared in either water for thrombin and trypsin substrates or 50% dimethylformamide for Suc-Ala-Ala-Pro-Phe-pNA. Measurements of the kinetic constants of Ala-Ala-Phe-pNA and homologous macrosubstrates were performed at 25° C. in order to allow measurements to be performed at substrate concentrations in excess above the Kₘ which decreased about 2-fold when the temperature was decreased from 37° C. to 25° C. These reactions were performed in 100 mM tris(hydroxymethyl)-aminomethane pH 7.8 with 10 mm CaCl₂. Substrate concentration were determined by absorbance at 342 nm, using an extinction coefficient of 8,260 mol⁻¹ and several concentrations were confirmed by absorbance measures at 405 or 410 nm after substrate hydrolysis, with extinction coefficient of 9,900 or 8,600 mol⁻¹, respectively, for the p-nitroaniline product (Lottenberg and Jackson, Biochim. Biophys. Acta 742:558-564, 1983). Molar concentration of chymotrypsin was determined by active site titration with p-nitrophenyl guanidinobenzoate and thrombin concentrations by titration with highly purified hirudin. Trypsin concentration was estimated based on weight, assuming 100% activity. For studies of trypsin and thrombin, α₂-macro-globulin was pretreated with a 200-fold molar excess of p-amidinophenylmethyl-sulfonylfluoride in order to inactivate any protease trapped within the inhibitor and left at 4° C. for 72 hours before use to allow complete decay of the sulfonylfluoride. Without this pretreatment inhibitor preparations had high peptidase activity for trypsin-type substrates.

B) Results

Synthesis and Size Analysis of Macrosubstrates

Covalent coupling of three pNA substrates (Succinyl-Ala-AlaPro-Phe-pNA, D-Phe-Pip-Arg-pNA, and D-Ile-Pro-Arg-pNA) with molecular weights of about 500 Da to PEG derivatives of 3,400 Da yielded products with a much larger hydrodynamic size than the substrate alone. The PEG derivatives possess two potential coupling sites, but reactions were performed so that monovalent products were expected to predominate.

The hydrodynamic sizes of macrosubstrates were determined by gel exclusion chromatography on Bio-Gel P-60. Elution of macrosubstrates and free substrate in separate runs were monitored at 318 nm for SucAlaAlaProPhe-p-nitroanilide, D-PhePipArg-p-nitroanilide, and D-IleProArg-p-nitroanilide, and the elution positions of macrosubstrates were plotted versus standards (ovalbumin (45,000 Da); trypsinogen (24,000 Da); trypsin inhibitor (20,000 Da); and aprotinin (6,500 Da)). Analysis of the three macrosubstrates by gel filtration by gel exclusion chromatography on a Bio-Gel P-60 column indicated that the hydrodynamic size of each conjugate was comparable to a protein of about 18,000 Da, corresponding to an effective radius in solution of 20 Å. There was a single major peak preceded by a minor component that may represent dimers of the PEG derivative.

The polymeric component of the PEGs represents a distribution of polymer lengths with a mean molecular weight of about 3,400 rather than a single defined polymer length and this may contribute to breadth of peaks. The peptide and PEG components of the macrosubstrates were joined by amide bonds that are stable in aqueous solution, and there was no detectable free substrate. It was not possible to estimate the hydrodynamic size of the free substrates in this analysis, because they adsorbed weakly on the column and eluted at slightly greater than the total column volume. The column adsorption of the free substrates reflects the hydrophobic character of pNA substrates, which in some cases require organic solvents such as dimethylsulfoxide to prepare concentrated stock solutions.

A favorable property of the macrosubstrates was high solubility in water, reflecting dominance of the PEG component on the physical characteristics of the macrosubstrate. Size estimates of macrosubstrates are consistent with previous estimates of the size of free PEG (Squire, Meth. Enzymol. 117:142-153, 1985; Bhat et al., Protein Sci 1:1 133-1143, 1992). PEGs have an extended random coil structure with a relatively large hydrodynamic size per molecular weight compared with globular proteins (Squire, Meth. Enzymol. 117:142-153, 1985; Bhat et al., Protein Sci 1:1 133-1143, 1992; Tarvers and Church, Int. J. Peptide Protein Res. 26:539-549, 1985). This explains how a macrosubstrate with a PEG of only 3,400 Da can model the size of a small globular protein of about 18,000 Da.

A homologous series of macrosubstrates of various sizes was prepared by reaction of methoxypolyethylene glycol (mPEG) derivatives of various polymer lengths with the substrate Ala-Ala-Phe-pNA. The size of the linear mPEG component varied from 1,000 to 21,000. The mPEG derivatives each have a single propionic acid (PA) group that serves as a coupling site for formation of an amide linkage to the N-terminus of the chromogenic substrate. The chemical nature of this series of substrates insures that all resulting macrosubstrates will be monofunctional, whereas some products of the bifunctional PEGs described above could have two substrate groups per molecule, depending on conditions of synthesis. A monofunctional macrosubstrate was also prepared with Ala-Ala-Phe-pNA linked to the C-terminus of a lysine residue that bears two mPEGs of about 5,500 Da. This simulates a macrosubstrate with the substrate site linked to the middle of PEG chain of 11,000 Da.

Analysis of the hydrodynamic sizes of the series of various sized macrosubstrates by gel exclusion chromatography on a column of Superose 12 indicated that the macrosubstrates had a range of hydrodynamic size corresponding to globular proteins of about 6,500, 12,000, 24,000 and 250,000 Da, respectively for products with mPEG components of 1,000, 1,800, 5,100, and 21,000 (Table 1). These results indicate that hydrodynamic radii of the macrosubstrates were similar to globular proteins of about 15, 17, 24, and 52 Å. The size estimates for macrosubstrates were generally consistent with published values for the size exclusion behavior of PEG components alone (Squire, Meth. Enzymol. 117:142-153, 1985), indicating that the peptide component had little contribution to hydrodynamic size. For these analyses gel filtration chromatography was performed with the addition of 10% acetonitrile in order to suppress hydrophobic adsorption of the macrosubstrates to the column. Adsorption to the column would have led to falsely low estimates of their molecular size. The free substrate adsorbed to the column even in the presence of 10% acetonitrile, and it eluted at slightly greater than a total column volume. Gel filtration of macrosubstrates on a column of Bio-Gel P-60 yielded similar size estimates for the three smaller macrosubstrates, but the largest macrosubstrate was beyond the measuring range of that gel filtration medium.

TABLE 1
Size analysis of a series of Ala-Ala-Phe-pNA substrates by
gel filtration chromatography on a column of Superose 12.
Sizes are estimated by comparison to globular protein standards.
K_{AV} is the calculated partition coefficient for molecules on
the column. Protein radii are from (Tarvers and Church, Int. J.
Peptide Protein Res. 26: 539-549, 1985).

Comparable Size Proteins

| | K_{AV} | MW (Da) | Radius (Å) |
|---|---|---|---|
| Macrosubstrate | | | |
| MPEG 1,000 | 0.703 | 6,500 | 15 |
| mPEG 1,800 | 0.63 | 12,000 | 17 |
| mPEG 5,100 | 0.52 | 23,800 | 24 |
| mPEG 21,500 | 0.26 | 250,000 | 52 |
| Protein Standards | | | |
| Aprotinin | 0.73 | 6,500 | 15 |
| Carbonic anhydrase | 0.50 | 29,000 | 24 |
| BSA | 0.37 | 66,000 | 35 |
| Catalase | 0.33 | 232,000 | 52 |
| Ferritin | 0.23 | 440,000 | 59 |

The greatest divergence of macrosubstrates size from that expected for the PEG component alone was for the largest substrate, which had an effective radius about 10% larger than expected from published values (Squire, Meth. Enzymol. 117:142-153, 1985; Bhat et al., Protein Sci 1:1133-1143, 1992). The macrosubstrate with a mPEG component of 21,000 Da also differed from the other macrosubstrates in that it did not yield a single major peak during gel filtration chromatography; the largest macrosubstrate gave a peak corresponding to the size of a globular protein of 250,000, as well as a series of peaks corresponding to smaller size products. The very long flexible polymer chain of this macrosubstrate may lead it to behave as an entangled polymer, and its solutions have been observed to precipitate as a gel in some cases, probably due to self-aggregation.

Analysis of the series of Ala-Ala-Phe-pNA macrosubstrates by reverse-phase BPLC, yielded single major peaks for each substrate, with a trace of free substrate detectable in some preparations. The free substrate had a retention time of 13.9 min versus 17.4, 17.3, 17.2 and 16.9 min respectively for the macrosubstrates with mPEG components of 1,000, 1,800, 5,100, and 21,000. The macrosubstrates were more strongly retained by the octadecylsilica matrix than was the free substrate, but the greater adsorption of the macrosubstrates probably was due to linkage of PA to the N-terminus of the peptide chain which substitutes a more hydrophobic amide for the free amino group. Adsorption of the macrosubstrates decreased slightly as the chain length of the mPEG increased, indicating that the mPEG component did not contribute to the adsorption of the macrosubstrate to the column. The peptide component probably served as the major element in adsorption to the column. Analysis of macrosubstrates with other peptide components showed widely varying retention. The slight decrease in adsorption with increasing mPEG size may reflect exclusion from some pores of the stationary phase even though a wide-pore silica was used.

Kinetics of Macrosubstrate Cleavage

Kinetic properties of small pNA substrates were compared versus macrosubstrates containing the same substrate sequences (Table 2). The vastly different elution of macrosubstrates versus their peptidyl-pNA components during gel filtration provided a simple means of purifying the macrosubstrates after reactions and obtaining material that was free of uncoupled substrate for kinetic analysis of substrate activity. Analyses as described above confirmed that there was essentially no unconjugated substrate in products. The maximal rates of cleavage (k_{cat}) of several substrates per molecule of chymotrypsin, trypsin, and thrombin decreased modestly when substrates were linked to PEG. The affinity of enzymes for substrates was affected more substantially. The Kₘs of macrosubstrates were higher than for the homologous free substrate—1.7-fold higher for a chymotrypsin substrate, 1.4 and 4.2-fold higher for two trypsin substrates, and 2.4 and 6.2-fold for two thrombin substrates. Even with the drop in affinity, Kₘs for all of the macrosubstrates were quite low—in the range from 10-110 μM. Primarily as a result of increase in Kₘ, efficiency of these substrates, measured as k_{cat}/Kₘ, decreased about 2 to 6-fold for proteinases such as trypsin and chymotrypsin which have a relatively accessible catalytic sites. Efficiency of cleavage of substrates by thrombin decreased by about 3-fold and 20-fold when a substrate was linked to PEG, possibly reflecting the greater steric hindrance of thrombin's active site or its highly selective extended substrate binding pocket. Most of the substantial loss of efficiency in thrombin's action on the substrate D-Phe-L-pipecolyl-L-Arg-pNA appeared to result from blocking the N-terminal amino group which is recognized to contribute to the efficiency of tripeptide thrombin substrates having D-amino acids at their N-terminus. Addition of an acetyl group to this substrate had an even greater effect than the addition of PEG. Thrombin macrosubstrates can continue to be optimized by reanalysis of the most favorable peptide sequence and evaluation of the most favorable linkages to PEG.

TABLE 2

Comparison of kinetic properties at 37° C. of peptidyl
substrates and homologous macrosubstrates with PEG of 3.400
Da. Values are the means of triplicates (PEG = polyethylene
glycol; Suc = succinyl; Ac = acetyl; Pip = L-pipecolyl).

| | | | | |
|---|---|---|---|---|
| | | kcat | Kₘ | k_{cat}/Kₘ |
| Protease | Substrate | (S⁻¹) | (uM) | (s⁻¹μM⁻¹) |

| | | | | |
|---|---|---|---|---|
| Chyrnotrypsin | Suc-Ala-Ala-Pro-Phe-pNA | 120 | 40 | 3.0 |
| | PEG-Suc-Ala-Ala-Pro-Phe-pNA | 100 | 68 | 1.5 |
| Trypsin | D-Ile-Pro-Arg-pNA | 53 | 8.7 | 6.1 |
| | PEG-Suc-D-Ile-Pro-Arg-pNA | 38 | 12 | 3.2 |
| Trypsin | D-Phe-Pip-Arg-pNA | 9.5 | 24 | 0.40 |
| | PEG-Suc-D-Phe-Pip-Arg-pNA | 8.3 | 110 | 0.075 |
| Thrombin | D-Ile-Pro-Arg-pNA | 105 | 12 | 8.7 |
| | PEG-Suc-D-Ile-Pro-Arg-pNA | 92 | 29 | 3.2 |
| | D-Phe-Pip-Arg-pNA | 65 | 6.1 | 11 |
| | Ac-D-Phe-Pip-Arg-pNA | 3.0 | 23 | 0.13 |
| | PEG-Suc-D-Phe-Pip-Arg-pNA | 23 | 38 | 0.61 |

Influence of Substrate Size on Efficiency

Unlike the examples in Table 2, some macrosubstrates have higher efficiency than the homologous free peptide substrate. The series of macrosubstrates prepared by linking Ala-Ala-Phe-pNA to mPEGs of various sizes showed a 40- to 80-fold improvement in substrate efficiency (k_{cat}/Kₘ) for chymotrypsin versus the free peptide substrate (Table 3). The macrosubstrates had both a substantially increased affinity for chymotrypsin and a higher turnover rate.

Preparation of the homologous series of macrosubstrates for chymotrypsin allowed the analysis of the effect of size on substrate efficiency independent of other structural issues. When the molecular weight of the macrosubstrates was increased by about 20-fold, it was observed that there an increase of Kₘ by about 40% and a decrease in k_{cat} of only about 15%. A macrosubstrate with the substrate group linked to a lysine residue between two mPEG chains of about 5,000 Da yielded a product that should have greater steric exclusion, and a two-fold lower affinity for chymotrypsin was observed relative to simple linear macrosubstrates with terminal substrate groups. A caveat in interpreting efficiency of this substrate is that the P4 substituent, in this case doubly-substituted Lys, may have influenced efficiency, but this example does point out the opportunity to employ branched macrosubstrates as further tools for probing steric factors in substrate-proteinase interactions.

TABLE 3

Kinetics of cleavage by chymotrypsin of Ala-Ala-Phe-pNA and
homologous macrosubstrates of various sizes as substrates of
chymotrypsin. Analyses were performed in triplicate at 25° C.
Values are means ± S.D (mPEG = methoxypolyethylene glycol;
PA = propionic acid).

| | | | |
|---|---|---|---|
| | k_{cat} | Kₘ | k_{cat}/Kₘ |
| Substrate | (s⁻¹) | (uM) | (s⁻¹μM⁻¹) |

| | | | |
|---|---|---|---|
| Ala-Ala-Phe-pNA | 2.2 ± 0.1 | 1.52 ± 0.22 | 1.4 |
| mPEG1,000-PA-Ala-Ala-Phe-pNA | 41.8 ± 0.4 | 0.36 ± 0.01 | 116 |
| mPEG1,800-PA-Ala-Ala-Phe-pNA | 43.4 ± 0.4 | 0.47 ± 0.01 | 92 |
| mPEG5,100-PA-Ala-Ala-Phe-pNA | 37.5 ± 3.2 | 0.51 ± 0.06 | 74 |
| mPEG21,500-PA-Ala-Ala-Phe-pNA | 35.3 ± 0.7 | 0.51 ± 0.02 | 69 |
| (mPEG5,000)₂-Lys-Ala-Ala-Phe-pNA | 35.9 ± 2.1 | 1.01 ± 0.08 | 36 |

Improved Specificity of Macrosubstrates for Proteinase Activity

We hypothesized that a major advantage of macrosubstrates relative to small chromogenic substrates would be the ability to distinguish between proteinase and peptidase activity. We examined this hypothesis using a model system that has been described in previous reports. α₂-Macroglobulin binds to and entraps proteinases without blocking their catalytic sites (Harpel and Mosesson, J. Clin. Invest. 52:2175-2184, 1973; Kueppers et al, Arch Biochem. Biophys 211:143-150, 1981; Barrett, Meth. Enzymol. 80:737-754, 1981; Sottrup-Jensen, J. Biol. Chem. 264:11539-11543, 1989; Qazi et al., J. Biol. Chem. 274:8137-8142, 1999; Mackie et al., Blood Coag Fibrinolysis 3:589-595). Consequently, protein substrates are sterically hindered from reaching catalytic sites and proteinase activity is nearly completely inhibited. Peptidase activity, however, is reported to be retained against peptides with a molecular weight less than about 8,000 that are still able to reach the catalytic site.

When increasing amounts of α₂-macroglobulin were added to a fixed amount of chymotrypsin prior to activity measurements, activity measured with a chromogenic substrate (SucAlaAlaProPhe-p-nitroanilide) was maximally inhibited by about 60% in the presence of excess α₂-macroglobulin, representing residual peptidase activity of the inhibitor-proteinase complex. Activity measured with the same substrate as a component of a macrosubstrate was inhibited by more than 95%.

Analysis of the inhibition of trypsin and thrombin activity by α₂-macroglobulin using pairs of substrate (D-Ile-Pro-Arg-p-nitroanilide (S-2288) as a substrate for trypsin and D-Phe-pipecolyl-Arg-p-nitroanilide (S-2238) as a substrate for thrombin) and macrosubstrate (PEG conjugates of the peptide substrates) yielded similar results. Activity of trypsin and thrombin measured with tripeptide substrates was inhibited about 40-50% by excess inhibitor. Activity of macrosubstrates incorporating the same peptide sequences was inhibited over 95%.

The relationship of macrosubstrate size to susceptibility to cleavage by proteinases complexed to α₂-macroglobulin was probed with the chymotrypsin substrates of various sizes (Table 4). Addition of an excess of α₂-macroglobulin to chymotrypsin led to a 43% inhibition of activity measured with a tripeptide substrate, and, respectively, inhibitions of 76%, 91% and 99% for macrosubstrates with mPEG components of 1,000, 1,800, and 5,100 Da. Results with the macrosubstrates with PEG of 3,400 Da, which yielded inhibition of about 95% above, are consistent with this size series. The different size macrosubstrates thus serve as a series of size probes to measure the accessibility of proteinases in the complex. Use of macrosubstrates with a PEG or mPEG component larger than 3,400 Da provides very low reactivity with complexed proteinase and could be used to selectively measure free proteinase in the presence of proteinase-α₂-macroglobulin complexes. Size estimates from macrosubstrate accessibility of the complexed proteinase are consistent with previous estimates that polypeptides larger than 8,000 Da are excluded from acting as substrates (Harpel and Mosesson, J. Clin. Invest. 52:2175-2184, 1973; Kueppers et al, Arch Biochem. Biophys 211:143-150, 1981; Barrett, Meth. Enzymol. 80:737-754, 1981).

TABLE 4

Inhibition of substrate cleavage by α-macroglobulin (α2-M).
Activity of chymotrypsin at a final concentration of 2.5 μg/ml
was measured before and after incubation with excess α2-macroglobulin
(250 μ/ml) using 0.5 mM substrate of various sizes.
Values are means ± SD of triplicate measurements.

Rate (μmol/L min)

| | | | |
|---|---|---|---|
| Substrate | Minus α₂-M | Plus α₂-M | Inhibition |

| | | | |
|---|---|---|---|
| Suc-Ala-Ala-Phe-pNA | 21.7 ± 0.2 | 12.3 ± 0.1 | 43% |
| mPEG1,000-PA-Ala-Ala-Phe-pNA | 28.2 ± 0.3 | 6.7 ± 0.1 | 76% |
| mPEG1,800-PA-Ala-Ala-Phe-pNA | 28.4 ± 0.7 | 2.6 ± 0.2 | 91% |
| mPEG5,100-PA-Ala-Ala-Phe-PNA | 23.9 ± 0.7 | 0.27 ± 0.01 | 99% |

### EXAMPLE II

Macromolecular Amylase Substrates: Effect of Substrate Size on Amylase Immunoinhibition Assays

To model the effects of substrate size on immunoinhibition assays, novel macromolecular substrates (macrosubstrates) for amylase were prepared by lining small chromogenic substrates to methoxypolyethylene glycol (mPEG). Gel filtration chromatography showed macrosubstrates to have a hydrodynamic radius of about 24 Å, similar to proteins of 30,000 Da. Macrosubstrates were good substrates for amylase. Polyclonal antisera versus amylase inhibited cleavage of macrosubstrates at several-fold lower antibody concentrations than cleavage of small substrates. Potency of inhibition also decreased according to chain length of small substrates (7>5>3 glucose subunits). We conclude that increasing substrate size can expand the target area on an enzyme upon which antibody binding will block substrate access. Accordingly, use of larger substrates often can benefit performance of enzyme immunoinhibition assays or screening for enzyme inhibitors.

A) Materials and Methods

Reagents

Purified human pancreatic and salivary amylases were purchased from Scripps Laboratories (San Diego, Calif.) and Sigma (St. Louis, Mo.), respectively. 2-chloro-p-nitrophenol-α-D-maltotrioside (G3ClpNP), as a dry powder, was provided by Genzyme Diagnostics (Cambridge, Mass.). “Liquid” G3ClpNP, in 2-(N-morpholino)ethanesulfonic acid (MES) buffer pH 6.0 containing 350 mmol/L sodium chloride, 6 mmol/L calcium acetate, 900 mmol/L potassium thiocyanate (KSCN), and 0.1% sodium azide, was purchased from Equal Diagnostics (Exton, Pa.). p-nitrophenyl-α-D-maltopentaoside (G5pNP) was from Calbiochem (La Jolla, Calif.). 4,6-O-ethylidene p-nitrophenyl-α-D-maltoheptaoside (EtG7pNP) and recombinant α-glucosidase from Saccharomyces cerevisiae were from Boehringer Mannheim (Indianapolis, Ind.). Amylopectin azure, p-nitrophenol standard solution, and rabbit albumin were from Sigma (St. Louis, Mo.). Polyclonal rabbit antiamylase immunoglobulin fractions were purchased from Sigma (St. Louis, Mo.) (7.2 mg/mL, antiserum 1) and Calbiochem (San Diego, Calif.) (10.7 mg/mL, antiserum 2). Two mouse MABs specific to salivary amylase, MAB 88E8 (8) and MAB 66C7 (37), were provided by Roche Molecular Biochemical (Penzberg, Germany).

mPEG-coupled G3ClpNP and EtG7pNP substrates were prepared by reacting mPEG 5000 isocyanate (Shearwater Polymers, Huntsville Ala.) with a ˜two-fold molar excess of the glycoside in dimethylformamide/10% diisopropylethylamine for 16 hours at room temperature. Pegylated substrates were purified by gel filtration on Sephadex G-50 in 0.1%acetic acid. Conjugation efficiencies of EtG7pNP and G3ClpNP were ˜30% and ˜10%, respectively, relative to the starting amount of glycoside.

Gel-filtration Analysis of mPEG-coupled Substrates

To analyze the hydrodynamic size of substrates by gel filtration chromatography, a 1.5 cm×25 cm column of Bio-Gel P-60 (Bio-Rad Laboratories, Hercules, Calif.) in 50 mmol/L sodium phosphate, pH 7.0 with 10% acetonitrile was used. Fractions of 0.8 ml were collected and absorbances at 305 nm were determined for substrates and at 280 nm for molecular weight standards. Molecular weight standards were purchased from Sigma (St. Louis, Mo.) and contained bovine serum albumin (66,000 Da), carbonic anhydrase (29,000 Da), cytochrome c (12,400 Da), and aprotonin (6500 Da).

Spectral Analysis of Uncoupled and mPEG-coupled Substrates

Substrates were diluted into HEPES buffer and absorbances were recorded between 250 nm and 500 nm. To determine the maximum amount of substrate that could be cleaved with amylase, substrates were incubated with 60 units/mL of salivary amylase. When EtG7pNP and mPEG-EtG7pNP were used, 4 units/mL of α-glucosidase were included in the incubation.

Amylase Assays

Amylase assays using oligosaccharide substrates were automated using a Cobas Fara analyzer (Roche, Basel, Switzerland). Unless indicated, assay temperature was 37° C., and absorbance changes were measured at 405 nm every minute over a 20 minute period. Assay buffer was either 52.5 mmol/L 4-(2-hydroxyethyl)-1-piperazine-ethanesulfonic acid (HEPES) buffer, pH 7.15, 87 mmol/L sodium chloride, 12.6 mmol/L magnesium chloride, 0.075 mmol/L calcium chloride (HEPES buffer) or 50 mmol/L MES, pH 6.0, 350 mmol/L sodium chloride, 6 mmol/L calcium acetate, 900 mmol/L KSCN (MES buffer). Amylases were diluted into assay buffer containing 1 mg/mL rabbit albumin. Final reaction volumes were 200 μL and consisted of 3 solutions, R1, R2, and R3. R1 contained buffer when G3ClpNP or mPEG-G3ClpNP were used as substrates and α-glucosidase (4 U/mL final concentration) when G5pNP, EtG7pNP, and mPEG-EtG7pNP were used as substrates. Unless indicated, final substrate concentrations were 0.8 mmol/L for G3ClpNP and mPEG-G3ClpNP and 0.4 mmol/Lfor G5pNP, EtG7pNP, and mPEG-EtG7pNP. Depending upon the assay, R2 and R3 contained either amylase or substrate. R1 and R2 solutions were mixed for 10 seconds prior to the addition of R3. For immunoinhibition studies, antibodies were preincubated with amylase (R2) for 30 minutes at room temperature prior to the addition of substrate (R3). Unless indicated, final concentrations of MABs were 10 mg/L. Reaction blanks were determined by measuring the absorbance of substrate or enzyme alone at 405 nm. When EtG7pNP or mPEG-EtG7pNP were used as substrates, α-glucosidase was included in the blank measurement. Differences between the test samples and reagent blanks were determined. Activity of stock solutions of amylase was based on activity measured with a Hitachi 917 analyzer using a Roche kit (Cat. No 1876473) (Indianapolis, Ind.) which uses EtG7pNP as the substrate. For Kₘ determinations, 4 or 5 different substrate concentrations (0.05 mmol/L to 6 mmol/L) with salivary (4-20 U/L) or pancreatic (4-40 U/L) amylase were used.

The assay using amylopectin azure was based on that of Rinderknecht et al. (Experientia 23:805, 1967). Assay buffer was 20 mmol/L sodium phosphate buffer, 50 mmol/L sodium chloride, pH 7.0 at 37° C. Following preincubation of amylase with antiamylase antibody in a volume of 100 μL for 30 minutes at room temperature, 900 μL of 2% (w/v) amylopectin azure was added, and the mixture was shaken at 37° C. for 1 hour. Reactions were stopped by the addition of 250 μL of 1 mol/L NaOH. Samples were centrifuged and absorbances of the supernatants were measured at 595 nm using a Lambda 4B UV/VIS spectrophotometer (Perkin-Elmer, Norwalk, Conn.). The absorbance of amylopectin azure substrate alone was subtracted from all values, and 100% activity was defined as salivary or amylase activity in the absence of any MAB

B) Results

Gel Filtration Analysis of mPEG-coupled Substrates

Bio-Gel P-60 elution profiles of mPEG-G3ClpNP, G3ClpNP, mPEG-EtG7pNP, and EtG7pNP, compared to calibration standards (bovine serum albumin (66 kDa); carbonic anhydrase (29 kDa); cytochrome C (12.4 kDa); and aprotinin (6.5 kDa)) showed that both mPEG-G3ClpNP and mPEG-EtG7pNP have size exclusion behavior similar to proteins of 30,000 Da. Uncoupled G3ClpNP and EtG7pNP eluted at slightly more than the total column volume, indicating that they adsorbed weakly on the column so that it was not possible to estimate the size of the free substrates by this technique. The analyses showed that the pegylated substrates did not contain any detectable free substrate. An additional smaller second peak of ˜60,000 Da was seen with mPEG-EtG7pNP and most likely represents EtG7pNP with two mPEG groups attached.

Extent of Cleavage of Uncoupled and mPEG-coupled Substrates by Amylase

To determine the maximal amount of mPEG-coupled and uncoupled substrate that could be cleaved by amylase, substrates were incubated with excess salivary amylase and the amount of free chromophore released was determined by measuring absorptions between 250 nm and 500 nm during amylase digestion. Absorbance spectra between 250 nm and 500 nm were measured in HEPES buffer containing 0.12 mmol/L G3ClpNP, 0.087 mmol/L mPEG-G3ClpNP, 0.12 mmol/L EtG7pNP, and 0.12 mmol/L mPEG-EtG7pNP alone or following digestion with salivary amylase and α-glucosidase (EtG7pNP and mPEG-EtG7pNP only). Incubations were monitored until endpoints of reactions were approached. α-Glucosidase was included in the incubation when EtG7pNP and mPEG-EtG7pNP were used as substrates.

G3ClpNP had a peak absorbance at 299 nm. Following digestion with salivary amylase, the peak at 299 nm disappeared and a peak at 400 nm, corresponding to free CNP, appeared. Using a molar extinction coefficient of 12,900 at 405 nm and pH 6.0 (package insert, Equal G3ClpNP Liquid Reagent), ˜90% of the CNP was released by salivary amylase after 40 minutes at room temperature. Coupling of G3ClpNP to mPEG caused a shift in the peak absorbance from 299 nm to 303 nm. Following digestion with salivary amylase, the peak at 303 nm decreased and a peak at 400 nm appeared. About 20% of the mPEG-G3ClpNP was cleaved by salivary amylase after 2 hours at room temperature.

EtG7pNP had a peak absorbance at 304 nm. Following digestion with salivary amylase and α-glucosidase for 60 minutes, this peak almost completely disappeared and a peak at 401 nm appeared. The absorbance spectra of pNP alone had a molar extinction coefficient of 11,300 at pH 7.15. Thus, ˜90% of EtG7pNP was cleaved by salivary amylase and α-glucosidase. Coupling of mPEG to EtG7pNP caused a shift in the peak absorbance from 304 nm to 292 nm and that following digestion with salivary amylase and α-glucosidase for 3 hours at room temperature, 50% of mPEG-EtG7pNP was cleaved.

Lag Phase

The time required to achieve constant reaction rates was studied with a Cobas Fara analyzer using purified pancreatic and salivary amylase and G3ClpNP, mPEG-G3ClpNP, G5pNP, EtG7pNP, and mPEG-EtG7pNP as substrates. G3ClpNP and mPEG-G3ClpNP showed little or no lag phase while G5pNP, EtG7pNP and mPEG-EtG7pNP required 3-4 min to reach maximal rates. Amylase activity was determined using absorbance changes in the linear segments of the curves.

Substrate Affinities for Amylase Isoenzymes

Table 5 lists the Kₘ values determined from Lineweaver and Burk plots. For the uncoupled substrates and similar to previous work (David, Clin. Chem. 28:1485-9, 1982), the Kₘ for the substrate decreased (affinity for amylase increased) as the number of chain length of substrates increased from 3 to 7 glucose units. Coupling of mPEG to EtG7pNP caused an increase in the Kₘ. Similar to EtG7pNP, coupling of mPEG to G3ClpNP resulted in an increase in the Kₘ. The Kₘ value for G3ClpNP was also determined in MES buffer pH 6.5 containing 300 mM KSCN. This caused a decrease in the Kₘ when compared with the HEPES buffer pH 7.15. A slight increase in the Kₘ was seen for G3ClpNP when the temperature was increased from 25° C. to 37° C. Previously published Kₘ values (David, Clin. Chem. 28:1485-9, 1982) for G7 (0.329 mmol/L with salivary amylase, 0.22 mmol/L with pancreatic amylase) and G5 (0.565 mmol/L with salivary amylase, 0.32 mmol/L with pancreatic amylase) substrates are similar to values described herein.

TABLE 5
Substrate affinities for amylase isoenzymes.
Kₘ values were determined as described in Materials and Methods.
Reactions were performed at 37° C. except where indicated.
Assays were in HEPES buffer pH 7.15 or, where indicated, at
pH 6.5 in MES buffer containing 300 mM KSCN.

| | Kₘ salivary | | Kₘ pancreatic | |
|---|---|---|---|---|
| substrate | amylase (mM) | | amylase (mM) | |
| EtG7pNP | 0.13 | | 0.17 | |
| mPEG-EtG7pNP | 0.89 | | 0.65 | |
| G5pNP | 0.40 | | 0.28 | |
| G3ClpNP | 6.9 | | 2.2 | |
| G3ClpNP | 4.6 | (25° C.) | 1.3 | |
| G3ClpNP | 2.0 | (pH 6.5) | 0.61 | (pH 6.5) |
| mPEG-G3ClpNP | 2.5 | (pH 6.5) | 1.8 | (pH 6.5) |

Effect of Substrate Size on Inhibition of Amylase Isoenzymes by Polyclonal Antisera

The effects of two different polyclonal antiamylase immunoglobulin preparations on salivary and pancreatic amylase activity using mPEG-coupled and uncoupled substrates were tested. Various volumes (0.01 μl, 0.1 μl, 1 μl, and 10 μl) of antiserum were preincubated with salivary or pancreatic amylase for 30 minutes before assay of activity with 0.8 mmol/L G3ClpNP, 0.4 mmol/L G5pNP, 0.4 mmol/L EtG7pNP, or 0.4 mmol/L mPEG-EtG7pNP. Final concentrations of salivary and pancreatic amylase were 20 U/I and 40 U/1, respectively. 100% amylase activity was determined in the absence of any antibody.

Both antisera cross-react with salivary and pancreatic amylases due to high sequence homology of the two isoenzymes, and the antisera inhibit amylase activity of both isoenzymes. The polyclonal antisera inhibited the cleavage of the mPEG-EtG7pNP and mPEG-G3ClpNP substrates by amylase at ˜2 and ˜10 fold lower antibody concentrations (points of 50% inhibition) than the corresponding uncoupled substrates, respectively. Potency of inhibition also decreased according to chain length of small substrates with cleavage of EtG7pNP being inhibited at >50 fold lower concentrations of antibody than G3ClpNP using salivary amylase and ˜3 fold lower concentrations of antibody using pancreatic amylase. Comparison of antibody inhibition curves for the largest substrate, mPEG-EtG7pNP, versus the smallest substrate, G3ClpNP, showed that curves were shifted 3 to 30 fold lower titers for the larger substrates in experiments with the two antisera and two isoenzymes. Because absorbance signals were smaller using mPEG-G3ClpNP as a substrate, 30 and 10 fold higher concentrations of salivary and pancreatic amylase, respectively, were used in order to directly compare G3ClpNP with mPEG-G3ClpNP. Using the smaller substrate about 10-fold more antibody was required to achieve equivalent inhibition to reactions with the larger substrate. However, a direct comparison of inhibition titers with other substrates is complicated because the higher enzyme concentrations shifted antibody inhibition curves to the right by ˜30 and ˜10 fold for salivary and pancreatic amylase, respectively.

Effect of MABs on Pancreatic and Salivary Amylase Activity Using mPEG-coupled and Uncoupled Substrates

Table 6 shows the percent of residual pancreatic and salivary amylase activity using MABs 88E8 and 66C7 for different substrates. MAB 88E8 inhibited 93-98% of salivary amylase with all substrates and did not significantly alter pancreatic amylase activity. MAB 66C7 did not substantially alter salivary or pancreatic amylase activity with any of the substrates. A synergistic effect is seen with all substrates using a mixture of MABs 88E8 and 66C7, and together these antibodies inhibit 95-99% of salivary amylase activity. No significant effect on pancreatic amylase activity was seen using a mixture of MABs 88E8 and 66C7. There was little difference in the magnitude of enzyme immunoinhibition when different substrates were used.

TABLE 6
Effect of MABs on salivary and pancreatic
amylase activity using various sized substrates.
Assays were performed as described in Materials and Methods. Final
amylase concentrations wer 20 U/I salivary and 40 U/I pancreatic for
G5 and G7 substrates and 600 U/I salivary and 400 U/I pancreatic for
G3 substrates. 100% activity was determined in the absence of MAB.

| | Amylase | % activity with MAB | | |
|---|---|---|---|---|
| Substrate | Isoenzyme | 66C7 | 88E8 | 88E8 + 66C7 |
| G3ClpNP | salivary | 104 | 3 | 1 |
| | pancreatic | 117 | 103 | 103 |
| PEG-G3ClpNP | salivary | 128 | 2 | 0.3 |
| | pancreatic | 104 | 101 | 101 |
| G5pNP | salivary | 105 | 6 | 2 |
| | pancreatic | 103 | 101 | 95 |
| EtG7pNP | salivary | 105 | 6 | 2 |
| | pancreatic | 105 | 96 | 96 |
| PEG-EtG7pNP | salivary | 100 | 7 | 5 |
| | pancreatic | 102 | 96 | 93 |

Concentration Curve with MABs 88E8 and 66C7 on Salivary Amylase Activity Using G3ClpNP as a Substrate.

The relative inhibition of salivary amylase activity by MABs 88E8 and 66C7 using G3ClpNP as a substrate was determined. Following preincubation of various concentrations (0, 2, 4, 6, 8, or 10 mg/L antibody) of MABs 88E8 and 66C7 with salivary amylase. in HEPES buffer pH 7.15, MES buffer pH 6.0 containing 900 mmol/L KSCN, or MES buffer pH 6.5 containing 300 mmol/L KSCN for 30 minutes, amylase activity was determined using G3ClpNP as substrate. When both MABs were used, MAB 88E8 concentrations were varied in the presence of 10 mg/L MAB 66C7. At an antibody concentration of 10 mg/L, MAB 88E8 inhibits 98% of salivary amylase activity in HEPES buffer and 34% of salivary amylase activity in MES buffer. MAB 66C7 increases salivary amylase activity in HEPES buffer and has no effect on salivary amylase activity in MES buffer. Using a mixture of MABs 88E8 and 66C7 at concentrations of 10 mg/mL each, >99% of salivary amylase activity is inhibited in HEPES buffer and 73% of salivary amylase activity is inhibited in MES buffer. When the pH of the MES buffer was increased from 6.0 to 6.5 and the concentration of KSCN was decreased from 900 mmol/L to 300 mmol/L, >97% of salivary amylase activity was inhibited by MAB 88E8.

Effect of MAB 66C7 on Amylase Activity Using Amylopectin Azure as a Substrate

The effect of increasing concentrations of MAB 66C7 on salivary and pancreatic amylase activity using amylopectin azure as a substrate was tested. Following pre-incubation of various concentrations of MAB 66C7 with 200 U/L salivary or 80 U/L pancreatic amylase for 30 minutes at room temperature, amylase activity was determined using amylopectin azure as described in the Materials and Methods section above. Salivary amylase activity was inhibited in a dose-dependent manner by this antibody and ˜50% salivary amylase activity was inhibited using 300 mg/L of MAB 66C7. Pancreatic amylase activity was unaffected by this antibody.

### EXAMPLE III

Novel Macromolecular Substrates for Thermolysin

Thermolysin is one of the best-characterized metalloproteases. Below are described novel macromolecular substrates for thermolysin in which small substrate peptides with a 3-(2-fir yl)acrylic acid (FA) amino-terminal blocking group are linked via their carboxyl-terminal end to methoxypolyethylene glycol (mPEG) amine. Examples of such substrates are FA-Gly-Leu-NH-mPEG and FA-Phe-Phe-NH-mPEG. The absorbance spectrum of the new substrates and the thermolysin cleavage products are very similar to corresponding amide substrates such as FA-Gly-Leu-amide. However, the new macromolecular substrates have several-fold higher efficiency, better solubility, and large molecular size that will allow analysis of steric factors in thermolysin action. Large size of the substrates also permits more sensitive detection of metalloprotease activity due the ease of separating small cleavage products with reporter groups from the intact substrate.

Metalloproteinases employ a metal ion such as zinc in thermolysin as an essential component of the catalytic site. Thermolysin and related metalloproteinases preferentially cleave on the amino-terminal side hydrophobic amino acid residues such as Leu and Phe. The cleavage specificity is further characterized by a minimal requirement for an amino-terminal blocked amino acid in the P1 position and an amidated amino acid in the P1′ position: R-Aaa ⇓ Aaa₂-amide, in which Aaa₂ is preferentially Leu, Phe, or another hydrophobic residue (Feder, J. and Schuck, J M. Biochemistry 9:2784-2791, 1970; Morihara, K. et al.. Eur J. Biochem. 15:374-380, 1970; and Morihara K. Meth. Enzymol. 248:242-253, 1995). Substrate efficiency increases substantially if peptide substrates are lengthened at their amino-terminal or carboxy-terminal ends, indicating the thermolysin has an extended substrate binding pocket that can interact with two or more residues preceding and following the cleavage site. An important consequence of the extended substrate requirements is that thermolysin will not cleave amide bonds formed by para-nitroaniline or other chromophores and fluorophores commonly used for monitoring the activity serine proteases.

The most common spectrophotometric method for monitoring activity of thermlolysin and related metalloproteases has been through use of FA substrates such as FA-Gly-Leu-amide as introduced by Feder (Biochem. Biophys. Res. Commun. 32:326-332, 1968). When cleaved after the P1 residue, there is a slight shift in the absorption spectrum to shorter wavelenths, resulting in a small decrease in absorbance at wavelengths between approximately 320 and 350 nm. Drawbacks of these substrates as models for protein cleavage are the very small size of peptide substrates which does not allow analysis of steric factors, small absorbance change per mole of product, low affinity, and limited solubility in water. We prepared two macromolecular substrates that overcome several of these limitations.

Materials and Methods.

Thermolysin (protease from Bacillus thermoproteolyticus, E.C. 3.4.24.2) was purchased from Sigma Chemical Co. (St. Louis, Mo.). Substrate spectra and cleavage were analyzed with a Cary 50 spectrophotometer at 25° C. in 50 mM HEPES pH 7.5, 10 mM CaCl₂. The peptides FA-Gly-Leu-amide, FA-Gly-Leu, FA-Phe-Phe, and FA-Phe were from Bachem Bioscience, Inc. (King of Prussia, Pa.). Methoxypolyethylene glycol (mPEG) amine with a molecular weight of approximately 5,000 was obtained from Shearwater Polymers (Huntsville, Ala.). FA-peptides were activated by diisopropylcarbodiimide in dichloromethane containing an equivalent amount of N-hydroxysuccinimide. After activation, the FA-peptides were coupled to mPEG-amine in dimethylformamide by methods similar to those previously described above and in Hortin, G. L., et al. Clin. Chem. 47:215-222, 2001. The polymer conjugates were separated from small reactants by passage through an anion-exchange Sephadex.

Results.

Absorbance spectra of the new substrates and the difference spectra following thermolysin action were virtually identical to the small amide substrates. This indicated that the macromolecular FA substrates were suitable for measuring thermolysin activity.

Rates of absorbance change with the pegylated substrates was about 4-10 fold greater than for FA-Gly-Leu-amide at substrate concentrations between 0.5 and 1 mM.

This suggests a substantially higher efficiency of the pegylated substrates, although it has not yet been established whether higher efficiency results improved affinity or higher turnover rates. Detailed comparison of the kinetics of cleavage of these substrates is complicated by the very high Kₘ of the amide substrate that exceeds its solubility. While not wishing to be bound by theory, initial results suggest that the mPEG-amine may weakly substitute for P2 residues that increase substrate affinity.

Discussion.

This study extends our previous studies of macromolecular substrates of serine proteases, described above, to show the feasibility of generating substrates for other classes of proteases and to make substrates with carboxy-terminal rather than amino-terminal addition of the polymeric component. The large size different between intact macromolecular substrates and the small fragments cleaved off by proteases should allow efficient separation of substrate and product and very sensitive detection of protease activity through detection of released absorbance, fluorescence, or luminescence, and the large size difference of substrate and product will likely allow the use of homogenous detection technologies such as fluorescence polarization.

Use of mPEG as a polymeric component is highly desirable due to the large effective size per molecular weight, availability in variable size, favorable solubility, and the defined chemical structure of substrates due to the unique coupling site of mPEG or two sites at the opposing ends of the linear polymer PEG (Hortin, G. L., et al. Clin. Chem. 47:215-222, 2001; and Harris, J. M. in Poly(ethylene glycol) Chemistry, Ed. J. M. Harris, Plenum Press, New York, 1992, pp. 1-14). The new thermolysin substrates offer the practical advantages of higher efficiency, improved solubility, and the potential for simplified stepwise synthesis on the polymer that is likely to serve as a simpler and less expensive route to preparing these substrates.

### EXAMPLE IV

Substrate Size Selectivity of 20S Proteasomes

Proteasomes are cytoplasmic complexes that have physiological importance as a major pathway for intracellular protein turnover and for generation of protein fragments for antigen presentation. The 20S proteasome, which is the core proteolytic component, has been demonstrated by X-ray crystallography to be a tubular structure that is highly conserved from eukaryotes to prokaryotes. The structure consists of two inner rings each with 7 β subunits sandwiched between two outer rings each with 7 α subunits. This tubular complex has a diameter of 110 Å, length of 150 Å, and a central hole about 50 Å. Catalytic sites are located on the luminal surface of proteasomes. Proteasomes do not belong to any of the four major classes of proteases—serine, cysteine, aspartate, or metalloproteases. An N-terminal threonine residue of β subunits appears to have a major catalytic role. Proteasomes have been termed multicatalytic proteinases due to the expression of multiple specificities including chymotrypsin-like, trypsin-like, and peptidylglutamyl-peptide hydrolyzing. Multiple specificities probably result from different types of β subunit. The location of catalytic sites within a tubular structure results in steric hindrance to the access of many substrates. The present study describes how substrate size serves as a limiting factor on rates of proteolysis by 20s proteasomes from Methanosarcina thermophila. Recombinant proteasomes from this organism have served as a useful model that has been studied extensively.

Substrate size was examined as an independent variable using macromolecular chromogenic substrates with a constant substrate group (Ala-Ala-Phe-p-nitroanilide) linked to variably-sized polyethylene glycol. Rates of substrate cleavage decreased progressively up to 10-fold as the substrate radius, estimated by gel filtration, increased from 15-50 Å. Cleavage of macromolecular substrates was saturable whereas cleavage of tripeptide substrates was not, and the smallest macromolecular substrates were more efficient substrates than free tripeptides. Thus, there appear to be mechanistic differences between the macromolecular and tripeptide substrates. We conclude that proteasomes serve as a size filter for selectively degrading substrates based on size and synthetic macromolecular substrates may serve as better tools than small substrates for measuring proteasome activity.

Materials and Methods

Materials. Recombinant Methanosarcina thermophila 20S proteasomes produced in Escherichia coli were purchased from Calbiochem (La Jolla, Calif.). The chromogenic substrates Ala-Ala-Phe-p-nitroanilide and succinyl-Ala-Ala-Phe-p-nitroanilide were from Bachem Bioscience, Inc. (King of Prussia, Pa.). Propionic acid (PA) derivatives of methoxypolyethylene glycol (mPEG) activated as N-hydroxysuccinimide esters were made by Shearwater Polymers (Huntsville, Ala.), which estimated average size of linear mPEGs to be 1,000, 1,800, 5,100, and 21,000 Da by gel permeation chromatography. Proteins for use as size standards were purchased from Sigma Chemical (St. Louis, Mo.).

Preparation of mPEG-substrate conjugates. Substrate synthesis was similar to that described above and in Hortin, G.L. et a l. Clin. Chem. 47:215-222, 2001. Ala-Ala-Phe-p-nitroanilide (100-200 mM) in dimethylformamide with 10% N-ethylmorpholine was mixed with 0.5-1.0 equivalent of mPEG active ester. After 2 h at room temperature, product was diluted with water and substrate conjugates were separated from free tripeptide substrate using a column of Sephadex G-25 in 0.2% acetic acid.

Molecular size analysis. Size exclusion chromatography was performed with a Pharmacia FPLC system using a 25 ml (1×31 cm) column of Superose. Void volume of the Superose 12 column was determined with blue dextran and the total column volume was determined with tritiated water. Elution of products was examined in 0.5 M NaCl and in 140 mM NaCl, 10 mM sodium phosphate pH 7.4 mixed with 10% or 30% acetonitrile to decrease the potential for adsorption to the column as noted for free substrates. Only slight differences were noted for elution of macrosubstrates in 10% and 30% acetonitrile, suggesting that there was little adsorption. Size comparisons of macrosubstrates versus protein markers was estimated from the linear relationship of log mw versus K_{AV}, the partition coefficient of the column which is (Ve-Vo)/(Vi-Vo), where Ve, Vo, and Vi are the elution, void, and included volumes of the column.

Enzyme assays. Assays of proteasome activity were performed at 37° C. unless otherwise indicated. Reactions had a total volume of100 μl reactions as described above and in Hortin, G. L. et al. (Clin. Chem. 47:215-222, 2001) using a Cobas FARA analyzer (Roche Diagnostic Systems, Somerville, N.J.). Reactions were in a buffer consisting of 100 mM Tris(hydroxymethyl)aminomethane, pH 7.2 with 1 mM dithiothreitol. Substrate concentration were determined with a Perkin-Elmer Lambda 4B spectrophotometer by absorbance at 342 nm, using an extinction coefficient of 8,250 mol⁻¹.

Results

Analysis of Effective Size of Synthetic Macromolecular Substrates.

The effective size of the conjugates of Ala-Ala-Phe-pNA with varying size mPEG were analyzed by gel filtration chromatography (Table 7). The effective radius of the synthetic macromolecular substrates ranged from 15-55 Å, depending on the size of the polymeric component. The effective radius of the macrosubstrates corresponded to values of globular proteins of 5-10 fold higher mass and approximated published values for the PEG components alone (Squire, P. G. Meth. Enzymol. 117:142-153, 1985). PEGs are recognized to behave as extended random coil polymers that have a large effective radius per mass (Squire, P. G., supra; and Harris, J. M. in Poly(ethylene glycol) Chemistry, Ed. J. M. Harris, Plenum Press, New York, 1992, pp. 1-14). Since the peptide sequence is kept constant in the homologous series of macrosubstrates, size can be examined as an independent variable in the efficiency of substrate cleavage.

Kinetics of Macrosubstrate Cleavage by Proteasomes.

When rates of cleavage of the macrosubstrates containing the sequence Ala-Ala-Phe-pNA were compared with that of an equimolar concentration (1 nm) of the homologous small substrate succinyl-Ala-Ala-Phe-pNA, it was observed the smallest macrosubstrate was cleaved at a rate about 6 fold greater than the small succinylated substrate (Table 8). There was a strong size dependence on the rate of macrosubstrate cleavage, however. The rate of cleavage decreased about 9-fold across the range of macrosubstrates from smallest to largest, which corresponded to an approximately 3-fold increase in effective radius of the macrosubstrates as assessed by gel filtration. Previous experiments have shown a much smaller effect of macrosubstrate size (a change of 30-50%) on rates of cleavage of proteases such as chymotrypsin and thrombin that are free in solution, as described above and in Hortin, G. L. et al. (Clin. Chem. 47:215-222, 2001). This probably reflects a much stronger steric limitation to the access of the active sites in proteasomes.

Further studies of the kinetics of substrate cleavage by proteasomes examined the cleavage of Ala-Ala-Phe-pNA, Suc-Ala-Ala-Phe-pNA, and the macrosubstrates with mPEG components of 1,000, 1,800, and 5,100 daltons at varying substrate concentrations from 0.5-3 mM and at varying temperatures from 30-50° C. The Suc-Ala-Ala-Phe-pNA was cleaved at a rate 50-100% greater than Ala-Ala-Phe-pNA. As the concentration of the small substrates increased, there was no evidence of saturation of reactions. In fact, reaction rates for the small substrates increased more than the proportional increase in concentration, suggesting activation of Proteasomes by the substrates. In contrast, the macrosubstrates showed saturation kinetics with Kₘ s of about 1 mM.

TABLE 7
Gel filtration of Ala-Ala-Phe-pNA
macrosubstrates compared with protein standards.

| | | Comparable Size Proteins | |
|---|---|---|---|
| | K_{AV} | MW (kDa) | Radius (Å) |
| Macrosubstrate | | | |
| mPEG 1,000 | 0.70 | 6.5 | 15 |
| mPEG 1,800 | 0.63 | 12 | 17 |
| mPEG 5,100 | 0.52 | 24 | 24 |
| mPEG 21,500 | 0.26-0.34* | 150-250 | 50-52 |
| Protein Standards | | | |
| Aprotinin | 0.73 | 6.5 | 15 |
| Carbonic anhydrase | 0.50 | 29 | 24 |
| BSA | 0.37 | 66 | 35 |
| Catalase | 0.33 | 232 | 52 |
| Ferritin | 0.23 | 440 | 59 |

Analysis on a Superose 12 column. K_{AV} is the partition coefficient on the column. Hydrodynamic radii of proteins are based on Tarvers, R. C. and Church, F. C. (Int. J. Peptide Protein Res. 26: 539-549, 1985).
*Broad size distribution suggesting some self-association.

TABLE 8

Rates of Substrate C1eavage by 20s Proteasomes.

| Substrate* | Rate (ΔA405/10 min, mean ± SD) |
|---|---|
| Suc-Ala-Ala-Phe-pNA | 0.0061 ± 0.0001 |
| mPEG 1,000-PA-Ala-Ala-Phe-pNA | 0.0371 ± 0.0006 |
| mPEG 1,800-PA-Ala-Ala-Phe-pNA | 0.0213 ± 0.0002 |
| mPEG 5,100-PA-Ala-Ala-Phe-pNA | 0.0095 ± 0.0003 |
| mPEG 21,000-PA-Ala-Ala-Phe-pNA | 0.0043 ± 0.0003 |

*Each substrate at 1 mM
PA = propionic acid

Discussion.

The present study revealed significant differences between the cleavage of Ala-Ala-Phe-pNA and macrosubstrates containing the same peptide sequence by 20s proteasomes from Methanosarcina thermophila. The kinetics of cleavage of the small substrate appeared to undergo substrate activation rather than saturation while larger substrates showed saturable kinetics. This may relate to the ability of several small substrate molecules simultaneously to occupy the lumen of a proteasome and to serve as allosteric modulators. In the case of the macrosubstrates, the lumen of the proteasome should be able to accommodate only one substrate molecule at a time. At low substrate concentrations, the macrosubstrates were cleaved more efficiently than small substrates. This was surprising in that large size should hinder access of substrates to the active sites of proteasomes. Clearly there was a strong steric effect in that larger macrosubstrates had progressively lower rates of cleavage. However, the macrosubstrates probably had a higher efficiency than the homologous small substrate due to a higher affinity for the catalytic sites. Proteasomes typically cleave proteins into peptides about 10 residues in length, suggesting that there may be a highly extended substrate binding pocket. The polymer chain of macrosubstrates may be able to substitute partially for an extended polypeptide chain of substrates. These observations suggest that synthetic macrosubstrates as described in the present application should serve as improved models of the uncoiled polypolypeptide chains that serve as physiological substrates of proteasomes. The macrosubstrates will allow analysis of steric factors in proteasome action, better kinetic modeling of the processive action of proteasomes on one substrate molecule at a time, and more sensitive monitoring of activity at low substrate concentrations.

Results of the present study extend previous descriptions of the applications of macrosubstrates in two important respects. First, Example I describes the use of macrosubstrates for measuring the activity of serine proteases, but the present study shows that macrosubstrates can be applied to measure the activity of other classes of proteases with different catalytic mechanisms. It is very likely that macrosubstrates can be developed for measuring the activity of any class of protease. Second, macrosubstrates have been applied to examine steric hindrance by the binding of an inhibitor to proteases (Example I and Hortin, G. L., et al. Clin. Chem. 47:215-222, 2001) and by the binding of antibodies (Example II and Warshawsly I, and Hortin G L. J. Clin Lab Anal 15:64-70, 2001). The present example shows how macrosubstrates can be applied to examine steric factors in the cleavage of substrates by proteases that consist of large macromolecular complexes. Many physiological proteases, including coagulation and complement factors, perform there primary physiological functions as part of large multicomponent complexes. Results of this study suggest that substitution of a macrosubstrate for small substrate can lead to substantial differences not only in the efficiency of substrate cleavage but also in the mechanisms of interaction between protease and substrate. In cases where physiological substrates are macromolecules, use of macrosubstrates rather than small substrates generally provides a closer representation of physiological reactions.

Incorporation by Reference

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of detecting a proteinase in a sample, comprising:
a) contacting the sample with a macrosubstrate for the proteinase, and
b) detecting the amount of macrosubstrate cleavage in the sample, whereby an increase in the amount of macrosubstrate cleavage detected in the sample, compared to the amount of macrosubstrate cleavage in a control sample lacking the proteinase, detects the proteinase in the sample.

2. A method of measuring the activity of a proteinase in a sample, comprising:
a) contacting the sample with a macrosubstrate for the proteinase, and
b) measuring the amount of macrosubstrate cleavage in the sample, whereby the amount of macrosubstrate cleavage measured in the sample, compared to the amount of macrosubstrate cleavage in a control sample, measures the activity of the proteinase in the sample.

3. The method of claim 1 or 2, wherein the proteinase is selected from a proteinase of the coagulation pathway, a proteinase of the fibrinolytic pathway, a proteinase of the complement pathway, a proteinase of an inflammatory pathway, or a proteinase of the digestive system.

4. The method of claim 3, wherein the proteinase is elastase.

5. The method of claim 1 or 2, wherein the proteinase is produced by a pathogen.

6. The method of claim 5, wherein the pathogen is a bacterium, a virus, or a fungus.

7. The method of claim 5, wherein the proteinase is activated by endotoxin.

8. The method of claim 1 or 2, wherein the proteinase is an asparty1 proteinase.

9. The method of claim 8, wherein the asparty1 proteinase is Human Immunodeficiency Virus (HIV) protease.

10. The method of claim 1 or 2, wherein the proteinase is a serine proteinase.

11. The method of claim 1 or 2, wherein the proteinase is a metalloproteinase.

12. The method of claim 1 or 2, wherein the proteinase is a proteasome proteinase.

13. The method of claim 2, wherein the control sample is negative for activity of the proteinase.

14. The method of claim 2, wherein the control sample is positive for activity of the proteinase.

15. A method of measuring amylase activity in a sample, comprising:
a) contacting the sample with an amylase macrosubstrate, and
b) measuring the amount of amylase macrosubstrate cleavage in the sample, whereby the amount of amylase macrosubstrate cleavage measured in the sample, compared to the amount of amylase macrosubstrate cleavage in a control sample, measures the amylase activity in the sample.

16. A method of diagnosing pancreatitis in a subject, comprising:
a) contacting a sample from the subject with an amylase macrosubstrate, and
b) measuring the amount of amylase macrosubstrate cleavage in the sample, whereby an increase in amylase macrosubstrate cleavage, relative to the amount of amylase macrosubstrate cleavage in a sample from a normal subject, diagnoses pancreatitis in the subject.

17. A method of detecting a target isoenzyme in a sample, comprising:
a) contacting the sample with an antibody that specifically binds to and inhibits the activity of a background isoenzyme;
b) contacting the sample with a macrosubstrate for the target isoenzyme; and
c) detecting the amount of macrosubstrate cleavage in the sample, whereby an increase in the amount of macrosubstrate cleavage detected in the sample, compared to the amount of macrosubstrate cleavage in a control sample lacking the target isoenzyme, detects the target isoenzyme in the sample.

18. A method of identifying a compound that modulates the activity of a proteinase, comprising:
a) exposing the proteinase to a macrosubstrate and to the compound, wherein the compound does not significantly bind the macrosubstrate; and
b) measuring the activity of the proteinase, whereby an increase or a decrease in the amount of macrosubstrate cleaved by the proteinase, relative to the amount of macrosubstrate cleaved by the proteinase not exposed to the compound, identifies a compound that modulates the activity of the proteinase.

19. A method of measuring the amount of heparin activity in a sample, comprising:
a) contacting the sample with a macrosubstrate for thrombin or factor Xa, and
b) detecting the amount of macrosubstrate cleavage in the sample, whereby the amount of macrosubstrate cleavage measured in the sample, compared to the amount of macrosubstrate cleavage in a control sample having a known amount of heparin activity measures the amount of heparin activity in the sample.

20. A method of measuring the amount of antithrombin III activity in a sample, comprising:
a) contacting the sample with a macrosubstrate for thrombin or factor Xa, and
b) detecting the amount of macrosubstrate cleavage in the sample, whereby the amount of macrosubstrate cleavage measured in the sample, compared to the amount of macrosubstrate cleavage in a control sample having a known amount of antithrombin III activity measures the amount of antithrombin III activity in the sample.

21. A method of measuring the amount of alpha-2-antiplasmin activity in a sample, comprising:
a) contacting the sample with a macrosubstrate for plasmin, and
b) detecting the amount of macrosubstrate cleavage in the sample, whereby the amount of macrosubstrate cleavage measured in the sample, compared to the amount of macrosubstrate cleavage in a control sample having a known amount of alpha-2-antiplasmin activity measures the amount of alpha-2-antiplasmin activity in the sample.

22. A method of inhibiting the activity of a proteinase, comprising contacting the proteinase with a macroinhibitor, thereby inhibiting the activity of the proteinase.
